(19) United States
(12) Patent Application Publication
(10) Pub. No.: US 2023/0165914 A1
(43) Pub. Date: Jun. 1, 2023

(54) THERAPEUTIC AND DIAGNOSTIC USE OF MICROORGANISMS FOR COVID-19

(71) Applicant: The Chinese University of Hong Kong, Shatin, New Territories (CN)

(72) Inventors: Siew Chien NG, Shatin, New Territories (CN); Ka Leung Francis CHAN, Tai Po, New Territories (CN); Tao ZUO, Qingzhou, Shandong (CN); Yun Kit YEOH, Kai Tak, KLN (CN)

(21) Appl. No.: 17/921,613

(22) PCT Filed: Apr. 28, 2021
(86) PCT No.: PCT/CN2021/090488

§ 371 Date: Oct. 26, 2022

Related U.S. Application Data
(60) Provisional application No. 63/016,759, filed on Apr. 28, 2020.
(60) Provisional application No. 63/025,310, filed on May 15, 2020.
(60) Provisional application No. 63/064,821, filed on Aug. 12, 2020.

(51) Int. Cl. A61K 35/745; A61K 35/747; A61P 31/14; A61K 45/06; C12Q 1/689

(52) U.S. Cl. CPC A61K 35/745; A61K 35/747; A61P 31/14; A61K 45/06; C12Q 1/689; C12Q 2600/118; C12Q 2600/16

(57) ABSTRACT

Methods are provided for treating COVID-19 patients to facilitate their recovery from the disease as well as for prognosis of severity of COVID-19 among patients who have been infected by SARS-CoV-2. Also provided are kits and compositions for use in these methods.

### RELATED APPLICATIONS

This application claims priority to U.S. Provisional Patent Application No. 63/016,759, filed Apr. 28, 2020, U.S. Provisional Patent Application No. 63/025,310, filed May 15, 2020, and U.S. Provisional Patent Application No. 63/064,821, filed Aug. 12, 2020, the contents of each of the above are hereby incorporated by reference in the entirety for all purposes.

### BACKGROUND OF THE INVENTION

In recent years, viral and bacterial infection is becoming more prevalent worldwide and presents a serious public health threat. For example, the Coronavirus-2019 (COVID-19) global pandemic of a respiratory disease caused by severe acute respiratory syndrome coronavirus 2 (SARS-CoV-2) has affected nearly 140 million people worldwide with close to 3 million deaths and is exacerbated by a large burden of asymptomatic carriers. Currently, experimental vaccines for COVID-19 are being tested globally in an attempt to prevent this disease or ameliorate its damaging effects to the patients, whereas various therapeutics are emerging and maturing for treating this disease and controlling its spread, especially the spread caused by the ever evolving variants of SARS-CoV-2. Thus, there exists an urgent need for new and meaningful treatment methods to control viral and bacterial infections as well as to lessen or eliminate their associated detrimental effects. The present invention fulfills this and other related needs by illustrating gut microbiota alterations and identifying various probiotic/prebiotics/therapeutic microorganism for the prevention and treatment of viral and bacterial infections.

### BRIEF SUMMARY OF THE INVENTION

The invention relates to novel methods and compositions useful for treating COVID-19 viral infection by the novel coronavirus SARS-CoV-2, such as in the prophylactic and therapeutic applications, including for facilitating recovery from the disease. In particular, the present inventor discovered that, as a result of infection by SARS-CoV-2, certain microorganism species, especially certain bacteria and viruses, are at an altered level, in the gastrointestinal (GI) tract of a COVID-19 patient. Health benefits such as prevention and alleviation of COVID-19 symptoms and detrimental effects can be achieved by modulating the level of pertinent microorganisms in patients' gut, for example, by fecal microbiota transplantation (FMT) treatment or oral administration of beneficial bacterial and/or viral species. These findings also provide new methods indicating the severity of COVID-19 in a patient. Thus, in the first aspect, the present invention provides a novel method for treating COVID-19, alleviating COVID-19 symptoms, and/or facilitating patient recovery from COVID-19, by increasing the level of Bacteroides dorei, or one or more bacterial species named in Table 4, 5, 6, 9, 13, or 18, or belonging to any one of the bacterial taxa set forth in Tables 19 and 21, or by increasing the level of one or more viral species named in Table 11.

In some embodiments, the method comprises the step of introducing into the subject's gastrointestinal tract an effective amount of one or more of the bacterial species of: Bacteroides dorei, or those set forth in Tables 4, 5, 6, 9, 13, and 18, or belonging to any one of the bacterial taxa set forth in Tables 19 and 21, and/or an effective amount of one or more of the viral species set forth in Table 11. For example, the introducing step comprises oral administration to the subject a composition comprising an effective amount of the desired bacterial species or viral species named above and herein. In some cases, the introducing step comprises delivery to the small intestine, ileum, or large intestine of the subject a composition comprising an effective amount of one or more of the desired bacterial species and/or comprising an effective amount of one or more of the desired viral species. In some embodiments, the introducing step comprises fecal microbiota transplantation (FMT). For instance, the FMT comprises administration to the subject a composition comprising processed donor fecal material, which may be by oral ingestion of the composition or by direct deposit the composition into the subject's gastrointestinal tract. In some cases, the introducing step further comprises simultaneously introducing to the subject a prebiotic or a therapeutic agent effective for treating COVID-19. In some embodiments, the prebiotic or therapeutic agent is introduced in the same composition comprising the effective amount of the desired bacterial species or viral species. In some embodiments, the composition is administered before and/or with food intake, especially in the case where the composition is formulated and packaged for oral administration. In some embodiments, the level or relative abundance of one or more of the bacterial species of Bacteroides dorei, or set forth in Tables 4, 5, 6, 9, 13, and 18, or belonging to any one of the bacterial taxa set forth in Tables 19 and 21, or one or more of the viral species set forth in Table 11 is determined in a first stool sample obtained from the subject prior to the introducing step and then again later in a second stool sample obtained from the subject after the introducing step. In some embodiments, the level of one or more of the bacterial species selected from Bacteroides dorei, those set forth in Tables 4, 5, 6, 9, 13, and 18, and belonging to any one of the bacterial taxa set forth in Tables 19 and 21, or the level of one or more of the viral species set forth in Table 11 is determined by quantitative polymerase chain reaction (PCR). In some embodiments, the one or more bacterial species comprise Bifidobacterium adolescentis, Faecalibacterium prausnitzii, Eubacterium rectale, Bifidobacterium bifidum, or Bifidobacterium longum, or any combination thereof.

In a second aspect, the present invention provides a method for treating COVID-19, alleviating COVID-19 symptoms, and/or facilitating patient recovery from COVID-19, by reducing the level, in the subject's gastrointestinal tract, of one or more bacterial species set forth in Tables 3, 7, 8, 12, and 17 or belonging to any one of the bacterial taxa set forth in Table 20, or one or more viral species set forth in Table 10.

In some embodiments, the reducing step comprises treating the subject with an anti-bacterial or anti-viral agent, which may be a broad-spectrum anti-bacterial or anti-viral agent, such as a broad spectrum antibiotic or anti-viral composition, or a specific anti-bacterial/anti-viral agent targeting one or more particular bacterial/viral species. In some embodiments, the reducing step comprising FMT, where a substance comprising processed donor fecal material is administered to the subject, e.g., by oral administration or by directly deposit into the subject's gastrointestinal tract. For example, the material has been processed such as dried, frozen or lyophilized, and placed in a capsule for oral ingestion. In some embodiments, a composition comprising processed donor fecal material is introduced to the gastrointestinal tract of the subject after the subject is treated with the anti-bacterial or anti-viral agent. In some embodiments, the method further comprises a step of simultaneously administering to the subject a prebiotic or a therapeutic agent effective for treating COVID-19, for example, the prebiotic or therapeutic agent is orally administered. In some embodiments, the level or relative abundance of the one or more of the bacterial species set forth in Table 3, 7, 8, 12, or 17, belonging to one or more of bacterial taxa set forth in Table 20, and/or the level or relative abundance of the one or more of the viral species set forth in Table 10 is determined in a first stool sample obtained from the subject prior to the reducing step and then again at a later time in a second stool sample obtained from the subject after the reducing step. In some embodiments, the level of the one or more bacterial species set forth in Table 3, 7, 8, 12, or 17, belonging to one or more of bacterial taxa set forth in Table 20, and/or the one or more viral species set forth in Table 10 is determined by quantitative polymerase chain reaction (qPCR). In some embodiments, the one or more bacterial species comprise Bifidobacterium dentium and/or Lactobacillus ruminis.

In a related aspect, a kit is provided for treating COVID-19 symptoms that comprises: a first container containing a first composition comprising (i) an effective amount of bacterial species Bacteroides dorei, or one or more of the bacterial species set forth in Tables 4, 5, 6, 9, 13, and 18, or belonging to any one of the bacterial taxa set forth in Tables 19 and 21, (ii) an effective amount of one or more of the viral species set forth in Table 11, (iii) an effective amount of an anti-bacterial agent that suppresses growth of one or more of the bacterial species set forth in Tables 3, 7, 8, 12, and 17 or belonging to any one of the bacterial taxa set forth in Table 20, or (iv) an effective amount of an anti-viral agent that suppresses growth of one or more of the viral species set forth in Table 10, and a second container containing a second composition comprising a prebiotic or a therapeutic agent effective for treating COVID-19, e.g., anti-viral agents such as ivermectin, or Zinc with quercetin or hydroxychloroquine, antibiotics such as azithromycin or doxycycline, vitamins such as C and D, as well as melatonin, or combinations thereof.

In some embodiments, the first composition comprises processed donor fecal material for FMT, for example, the material has been processed such as dried, frozen or lyophilized, and placed in a capsule for oral ingestion, or the material may be formulated for direct deposit in the subject's gastrointestinal tract. In some embodiments, the first composition is formulated for oral administration. In some embodiments, the second composition is formulated for oral administration. In some embodiments, both the first and the second compositions are formulated for oral administration.

In a third aspect, a method is provided for predicting severity of COVID-19 among patients who have been infected by SARS-CoV-2 by comparing the level of one or more bacterial species set forth in Table 2 or 6 and/or the level of one or more viral species set forth in Table 4 found in patient's gastrointestinal tract or in the stool sample. The method includes these step: determining, in a stool sample from a first human subject infected by SARS-CoV-2, the level or relative abundance of any one of the bacterial species set forth in Tables 6, 9, 13, and 18 or belonging to the bacterial taxa set forth in Tables 19 and 21, or the level or relative abundance of any one of the viral species set forth in Table 11; detecting the level of relative abundance from step (1) being higher than the level or relative abundance of the same bacterial or viral species in a stool sample from a second human subject infected by SARS-CoV-2; and determining the second subject as likely to experience more severe COVID-19 than the first subject.

In some embodiments, the level or relative abundance of multiple bacterial species set forth in Tables 6, 9, 13, and 18 or belonging to the bacterial taxa set forth in Tables 19 and 21 or multiple viral species set forth in Table 11 is determined, and the level or of more than half of the multiple bacterial or viral species in the first subject's sample is higher than the corresponding level or relative abundance in the second subject's sample, and the second subject is determined to likely experience more severe COVID-19 than the first subject. In some embodiments, the level or relative abundance of the bacterial or viral species is determined by quantitative PCR. In some embodiments, the method further comprises the step of administering to the second subject an effective amount of a therapeutic agent effective for treating COVID-19. In some embodiments, the bacterial species includes Bifidobacterium adolescentis, Faecalibacterium prausnitzii, Eubacterium rectale, Bifidobacterium bifidum, or Bifidobacterium longum, or any combination thereof.

In a four aspect, a method is provided for predicting severity of COVID-19 among patients who have been infected by SARS-CoV-2 by comparing the level of bacterial species set forth in Tables 1 and 5 or the level of viral species set forth in Table 3 found in patient's gastrointestinal tract. The method includes these step: determining, in a stool sample from a first human subject infected by SARS-CoV-2, the level or relative abundance of one or more of the bacterial species set forth in Tables 7, 8, 12, and 17 or belonging to one or more of the bacterial taxa set forth in Table 20, or the level or relative abundance of one or more of the viral species set forth in Table 10; detecting the level of relative abundance from step (1) being higher than the level or relative abundance of the same bacterial/viral species in a stool sample from a second human subject infected by SARS-CoV-2; and determining the first subject as likely to experience more severe COVID-19 than the second subject.

In some embodiments, the level or relative abundance of multiple bacterial/viral species set forth in Tables 7, 8, 10, 12, and 17 or belonging to the bacterial taxa set forth in Table 20 is determined, and the level or of more than half of the multiple bacterial/viral species in the first subject's sample is higher than the corresponding level or relative abundance in the second subject's sample, and the first subject is determined to likely experience more severe COVID-19 than the second subject. In some embodiments, the level or relative abundance of the bacterial or viral species is determined by quantitative PCR. In some embodiments, the method further comprises the step of administering to the first subject an effective amount of a therapeutic agent effective for treating COVID-19. In some cases, the bacterial species is Bifidobacterium dentium and/or Lactobacillus ruminis.

In a related aspect, the present invention provides a kit for assessing severity of COVID-19 comprising a set of oligonucleotide primers for amplification of a polynucleotide sequence from (1) any one of the bacterial species set forth in Tables 6, 7, 8, 9, 12, 13, 17, and 18 or belonging to any one of the bacterial taxa set forth in Tables 19-21, or (2) any one of the viral species set forth in Tables 10 and 11. In some embodiments, the amplification is PCR. In some embodiments, the kit further comprises reagents for quantitative PCR. For example, the bacterial species may include Bifidobacterium adolescentis, Faecalibacterium prausnitzii, Eubacterium rectale, Bifidobacterium bifidum, or Bifidobacterium longum or any combination thereof; or the bacterial species may include Bifidobacterium dentium and/or Lactobacillus ruminis.

### BRIEF DESCRIPTION OF THE DRAWINGS

FIG. 1 Timeline of symptoms onset, SARS-CoV-2 test, hospitalization and stool sample collection.

FIG. 2 Gut microbiome diversity and richness alterations in COVID-19 patients. (A) baseline microbiome diversity and richness in COVID-19 patients, compared to healthy controls and pneumonia controls. (B) longitudinal alterations in microbiome diversity and richness in COVID-19 patients.

FIG. 3 NMDS plot of the gut microbiomes across COVID-19, healthy controls, and pneumonia controls.

FIG. 4 (A) correlation plot between the bacteria species Rothia mucilaginosa and Bacteroides dorei and fecal viral load. (B) longitudinal dynamics of bacteria species Rothia mucilaginosa and Bacteroides dorei in COVID-19 patients.

FIG. 5 Bacteria significantly associated with fecal viral load during disease course, as determined by spearman correlation test.

FIG. 6 Compositional differences in gut microbiota microbiota composition between hospitalized COVID-19 patients and non -COVID-19 individuals. (A) Gut microbiota summarized at the phylum level. Values indicate mean±standard deviation. (B) Principal component analysis (PCA) of gut microbiota composition of COVID-19 patients with and without antibiotics compared with non-COVID-19 individuals. The p-value indicates a significant association between community composition and cohort (COVID-19 vs non-COVID-19) as well as antibiotics use (permutational multivariate analysis of variance). (C) Number of unique species and (D) Shannon diversity index of gut microbiotas of COVID-19 and non-COVID-19 individuals. Black lines in each box represent median values, top and bottom box boundaries represent upper and lower quartiles, respectively, and whiskers represent 1.5× interquartile range.

FIG. 7 Principal component analysis (PCA) of gut microbiota composition in COVID-19 patients and associations with plasma concentrations of cytokines. Filled circles represent community composition of the first stool samples (if serial samples are available) of hospitalized patients; circles are colored by disease severity classifications based on Wu et al (2020). The ellipses represent groupings of samples by disease severity category, where centroid of the four groups are indicated by the placement of their respective labels (mild, mod, severe, critical). Red arrows represent the direction of greatest linear increase in the gradients of measured cytokines fitted onto the gut composition PCA. Length of arrows reflect degree of correlation. Only cytokines measurements significantly associated with gut microbiota composition are shown (p<0.05, Procrustes analysis). The p-value in the bottom right of the panel indicates significant association between community composition and disease severity classification as indicated by permutational multivariate analysis of variance.

FIG. 8 Correlations between COVID-19 enriched/depleted gut microbial taxa and plasma cytokine concentrations. (A) CXCL10, (B) IL-10, (C) TNF-α, (D) CCL2, (E) CXCL8, (F) IL -1β, and (G) IL-6. Only statistically significant correlations are shown. Blue lines in each scatter plot represent the linear regression line, and shaded regions represent 95% confidence intervals.

FIG. 9 Gut microbiota composition of COVID-19 patients after negative SARS SARS-CoV-2 quantitative reverse transcription polymerase chain reaction (qRT-PCR) tests. (A) Principal component analysis of gut microbiota composition in recovered COVID-19 patients with and without antibiotics compared with non-COVID-19 individuals. (B) Average relative abundances of four beneficial gut bacteria in recovered COVID-19 patients compared with non-COVID-19 individuals. (C) Number of days from onset of COVID-19 symptoms until discharge from hospital between COVID-19 patients with and without antibiotics use. Black lines in each box represent median values, top and bottom box boundaries represent upper and lower quartiles, respectively, and whiskers represent 1.5× interquartile range.

FIG. 10 Principal component analysis (PCA) of gut microbiota composition in COVID-19 patients and associations with blood inflammation markers. Filled circles represent community composition of the first stool samples of hospitalized patients; circles are coloured by disease severity classifications based on Wu et al (2020). The ellipses represent groupings of samples by disease severity category, where centroid of the four groups area indicated by the the placement of their respective labels (mild, severe, critical). Red arrows represent the direction of greatest linear increase in the gradients of measured blood inflammation markers fitted onto the gut composition PCA. Length of arrows reflect degree of correlation. Only markers significantly associated with gut microbiota composition are shown (p<0.05, Procrustes analysis). The p-value in the bottom right of the panel indicates significant association between community composition and disease severity classification as indicated by permutational multivariate analysis of variance. AST: aspartate aminotransferase; CRP: C-reactive protein; ESR: erythrocyte sedimentation rate; GGT: gamma-glutamyl transferase; LDH: lactate dehydrogenase; NT-proBNP: N-terminal-pro-brain natriuretic peptide.

FIG. 11 Principal component analysis ordination showing spread of gut microbiota composition of all 196 stool samples collected from 101 COVID-19 patients during hospitalization and after negative SARS-CoV-2 quantitative reverse transcription polymerase chain reaction (qRT-PCR) tests. Data points are coloured by the respective patients' disease severity classification, and grouped according to whether patients received antibiotics (abx+ vs abx−) during their hospitalization.

### Definitions

The term “fecal microbiota transplantation (FMT)” or “stool transplant” refers to a medical procedure during which fecal matter containing live fecal microorganisms (bacteria, fungi, viruses, and the like) obtained from a healthy individual is transferred into the gastrointestinal tract of a recipient to restore healthy gut microflora that has been disrupted or destroyed by any one of a variety of medical conditions, for example, COVID-19. Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing processed (e.g., dried and frozen/lyophilized) fecal material.

The term “inhibiting” or “inhibition,” as used herein, refers to any detectable negative effect on a target biological process, such as RNA/protein expression of a target gene, the biological activity of a target protein, cellular signal transduction, cell proliferation, and the like. Typically, an inhibition is reflected in a decrease of at least 10%, 20%, 30%, 40%, 50%, 60%, 70%, 80%, 90% or greater in the target process (e.g., growth or proliferation of a microorganism of certain species, for example, one or more of the bacterial species shown in Table 3, 8, 12, or 17 or belonging to the bacterial taxa set forth in Table 20 or one or more of the viral species shown in Table 10), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” “reduction,” “decrease,” “decreasing,” “lower,” and “less” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” “enhancement,” “higher,” and “more” are used in this disclosure to encompass positive changes at different levels (e.g., at least 10%, 20%, 30%, 40%, 50%, 60%, 70%, 80%, 90%, 100%, 200%, or greater such as 3, 5, 8, 10, 20-fold increase compared to a control level, for example, the control level of one or more of the bacterial species shown in Table 2 or 6) in a target process or signal. In contrast, the term “substantially the same” or “substantially lack of change” indicates little to no change in quantity from a comparison basis (such as a standard control value), typically within ±10% of the comparison basis, or within +5%, 4%, 3%, 2%, 1%, or even less variation from the comparison basis.

The term “anti-bacterial/viral agent” refers to any substance that is capable of inhibiting, suppressing, or preventing the growth or proliferation of bacterial or viral species, respectively, especially those of shown in Tables 3, 8, 12, and 17 or Table 10, respectively. Known agents with anti-bacterial activity include various antibiotics that generally suppress the proliferation of a broad spectrum of bacterial species as well as agents such as antisense oligonucleotides, small inhibitory RNAs, and the like that can inhibit the proliferation of specific bacterial species. The term “anti-bacterial/viral agent” is similarly defined to encompass both agents with broad spectrum activity of killing virtually all species of bacteria/viruses and agents that specifically suppress proliferation of target bacteria/virus species. Such specific anti-bacterial/viral agent may be short polynucleotide in nature (e.g., a small inhibitory RNA, microRNA, miniRNA, lncRNA, or an antisense oligonucleotide) that is capable of disrupting the expression of a key gene in the life cycle of a target bacterial or viral species and is therefore capable of specifically suppressing or eliminating the species only without substantially affecting other closely related bacterial or viral species.

“Percentage relative abundance,” when used in the context of describing the presence of a particular bacterial or viral species (e.g., any one of those shown in any one of Tables 3-13, 17, and 18 or belonging to bacterial taxa set forth in Tables 19-21, or Tables 10 and 11, respectively) in relation to all bacterial or viral species present in the same environment, refers to the relative amount of the bacterial/viral species out of the amount of all bacterial/viral species as expressed in a percentage form. For instance, the percentage relative abundance of one particular bacterial species can be determined by comparing the quantity of DNA specific for this species (e.g., determined by quantitative polymerase chain reaction) in one given sample with the quantity of all bacterial DNA (e.g., determined by quantitative polymerase chain reaction (PCR) and sequencing based on the 16s rRNA sequence) in the same sample.

“Absolute abundance,” when used in the context of describing the presence of a particular bacterial/viral species (e.g., any one of those shown in Tables disclosed herein) in the feces, refers to the amount of DNA derived from the bacterial or viral species out of the amount of all DNA in a fecal sample. For instance, the absolute abundance of one bacterium or virus can be determined by comparing the quantity of DNA specific for this bacterial or viral species (e.g., determined by quantitative PCR) in one given sample with the quantity of all fecal DNA in the same sample.

“Total bacterial/viral load” of a fecal sample, as used herein, refers to the amount of all bacterial/viral DNA, respectively, out of the amount of all DNA in the fecal sample. For instance, the absolute abundance of bacteria can be determined by comparing the quantity of bacteria-specific DNA (e.g., 16s rRNA determined by quantitative PCR) in one given sample with the quantity of all fecal DNA in the same sample.

As used herein, the term “severe acute respiratory syndrome coronavirus 2 (SARS-CoV-2),” refers to the virus that causes Coronavirus Disease 2019 (COVID-19). It is also referred to as “COVID-19 virus.”

The term “treat” or “treating,” as used in this application, describes an act that leads to the elimination, reduction, alleviation, reversal, prevention and/or delay of onset or recurrence of any symptom of a predetermined medical condition. In other words, “treating” a condition encompasses both therapeutic and prophylactic intervention against the condition, including facilitation of patient recovery from the condition. The term “effective amount,” as used herein, refers to an amount of a substance that produces a desired effect (e.g., an inhibitory or suppressive effect on the growth or proliferation of one or more detrimental bacterial or viral species (e.g., the bacterial species shown in Tables 3, 8, 12, and 17 or belonging to the bacterial taxa set forth in Table 20, or the viral species shown in Table 10) for which the substance (e.g., an anti-bacterial/viral agent) is used or administered. The effects include the prevention, inhibition, or delaying of any pertinent biological process during bacterial/viral proliferation to any detectable extent. The exact amount will depend on the nature of the substance (the active agent), the manner of use/administration, and the purpose of the application, and will be ascertainable by one skilled in the art using known techniques as well as those described herein. In another context, when an “effective amount” of one or more beneficial or desirable bacterial or viral species (e.g., Bacteroides dorei, or those listed in Table 4, 5, 9, 13, or 18, or belonging to a bacterial taxa set forth in Table 19 or 21, or viruses set forth in Table 11) are artificially introduced into a composition intended to be introduced into the gastrointestinal tract of a patient, e.g., to be used in FMT, it is meant that the amount of the pertinent bacteria and/or virus(es) being introduced is sufficient to confer to the recipient health benefits such as reduced recovery time or reduced needs for therapeutic intervention for a pertinent disease such as COVID-19, including but not limited to medication, hospitalization, or more aggressive intervention such as ventilation and induced coma.

The term “severity” of a disease refers to the level and extent to which a disease progresses to cause detrimental effects on the well-being and health of a patient suffering from the disease, such as short-term and long-term physical, mental, and psychological disability, up to and including death of the patient. Severity of a disease can be reflected in the nature and quantity of the necessary therapeutic and maintenance measures, the time duration required for patient recovery, the extent of possible recovery, the percentage of patient full recovery, the percentage of patients in need of long-term care, and mortality rate. For example, reduced disease severity for a COVID patient may be manifested in the faster resolution of symptoms (such as cough, fever, chills, headache, full body pain, joint and/or muscle pain, loss of taste/smell, nausea, diathermia, etc.), including symptoms that persist beyond 2 weeks or 4 weeks after a COVID patient is PCR-negative for SARS-CoV-2.

As used herein, the term “about” denotes a range of value that is +/−10% of a specified value. For instance, “about 10” denotes the value range of 9 to 11 (10 +/−1).

### DETAILED DESCRIPTION OF THE INVENTION

### I. Introduction

The invention provides a novel approach for assessing the likely severity of COVID-19 among patients infected by severe acute respiratory syndrome coronavirus 2 (SARS-CoV-2) as well as for treating COVID-19 symptoms or facilitating patient recovery from COVID-19. During their studies, the present inventors discovered that the presence and relative abundance of certain bacterial and viral species alter significantly in the gastrointestinal tract of patients due to SARS-CoV-2 infection, with increase or decrease of particular species correlating with disease severity. For example, the presence of bacterial species shown in Table 3 is found to be at an elevated level in the gastrointestinal tract of COVID-19 patients, whereas the presence of bacterial species such as Bacteroides dorei and those shown in Table 4 or 5 have been found to be at a reduced level. Similarly, the reduced presence or level of certain bacterial species (such as those in Table 6) and/or the increased presence or level of certain bacterial species (such as those in Table 7) in COVID-19 patients' stool samples has been observed to correlate with likely more severe form of the disease with likely worse outcome and/or higher likelihood of requiring more extensive medical treatment and longer recovery time. As further examples, the level of bacterial species shown in Table 12 in the GI tract of a COVID-19 patient is found to correlate with the coronavirus load in the patient, whereas the level of bacterial species shown in Table 13 has been found to inversely correlate with patient's coronavirus load, whereas the reduced presence or level of certain bacterial and viral species (such as those in Table 9 and Table 11, respectively) and/or the increased presence or level of certain bacterial and viral species (such as those in Table 12 and Table 10, respectively) in COVID-19 patients' stool samples has been observed to correlate with likely more severe form of the disease with likely worse outcome and/or higher likelihood of requiring more extensive medical treatment and longer recovery time. Thus, the results of this study provide useful tools for assessing disease status and for aiding patient recovery from the infection by this novel coronavirus.

### II. FMT Donor/Recipient Selection and Preparation

COVID-19 Patients suffer from a disrupted state of GI tract microflora are considered as recipients for FMT treatment in order to restore the normal healthy profile for microorganisms. As revealed by the present inventors, the relative abundance of certain bacterial species/taxa and viral species such as those shown in Tables 4-13 and 17-21 correlate with the severity of COVID-19 or the coronavirus load in COVID-19 patients, a FMT donor whose fecal material contains an higher than average level of the bacterial or viral species in Tables 4-6, 9, 11, 12, 13, 18, 19, and 21 is favored as particularly advantageous for this purpose. For example, a desirable donor may preferably have higher than about 0.1% or up to about 10% of total bacteria in relative abundance for any one of these bacterial species in his stool sample.

On the other hand, COVID-19 patients with high level of the bacterial species/taxa or viral species listed in Table 3, 7, 8, 10, 12, 17, or 20 tend to suffer from a more severe form of the disease or have a higher virus load. Thus, to restore their normal and healthy GI bacterial profile, FMT is appropriate using fecal material donated from a healthy person whose level of these bacterial species/taxa (e.g., in Tables 3, 8, 12, 17, and 20) or viral species (e.g., in Table 10) in the stool sample is either naturally low or artificially depressed, for example, by the use of a specific anti-bacterial or viral agent that specifically kills or suppresses certain target bacterial or viral species without significantly impacting other bacterial or viral species. Preferably, each of these bacterial or viral species should have no more than about 0.01% of total bacteria or viruses in relative abundance in the fecal material before being processed for use in FMT.

Fecal matter used in FMT is obtained from a healthy donor and then processed into appropriate forms for the intended means of delivery in the upcoming FMT procedure. While a healthy individual from the same family or household often serves as donor, in practicing the present invention the donor microorganism profile is an important consideration and may favor the choice of an unrelated donor instead. The process of preparing donor material for transplant includes steps of drying, freezing or lyophilizing, and formulating or packaging, depending on the precise route of delivery to recipient, e.g., by oral ingestion or by rectal deposit.

In preparation for FMT treatment, an intended recipient, e.g., a patient who has been diagnosed with SARS-CoV-2 infection or who was diagnosed of COVID-19 but has been recently cured of the disease (e.g., was PCR-positive in viral nucleic acids and then became PCR-negative in the past 1-7 days), may first receive a treatment to suppress bacterial level in his GI tract prior to FMT. The treatment may involve administration of an anti-bacterial/viral agent, either a broad spectrum agent or a specific anti-bacterial/viral agent, to eliminate or reduce the level of undesirable bacterial or viral species that positively correlate with SARS-CoV-2 virus load or severity of the disease, such as one or more of the bacterial species or taxa named in Tables 4-6, 9, 13, 18, 19, and 21 or one or more of the viruses named in Table 11.

Various methods have been reported in the literature for determining the levels of all bacterial species in a sample, for example, amplification (e.g., by PCR) and sequencing of bacterial polynucleotide sequence taking advantage of the sequence similarity in the commonly shared 16S rRNA bacterial sequences. On the other hand, the level of any given bacterial species may be determined by amplification and sequencing of its unique genomic sequence. A percentage abundance is often used as a parameter to indicate the relative level of a bacterial species in a given environment. The level and relative abundance of viral species can be determined similarly using well-established methods in the pertinent research field.

### III. Treatment Methods by Modulating Bacteria or Virus Level

The discovery by the present inventors reveals the direct correlation between SARS-CoV-2 virus load or disease severity and the increase or decrease of certain bacterial species/taxa or viral species level (e.g., Bacteroides dorei and those shown in Tables 3-13 and 17-21) in COVID-19 patient's gut. This revelation enables different methods for treating COVID-19 symptoms, especially for aiding COVID-19 patients recover from the disease, by adjusting or modulating the level of these bacterial or viral species in these patient's GI tract via, e.g., an FMT procedure, to either deliver to the patients' GI tract an effective amount of one or more of the bacterial species/taxa or viral species shown in Tables 4-6, 9, 11, 13, 18, 19, and 21 or Bacteroides dorei to decrease the level of one or more bacterial species/taxa or viral species listed in Table 3, 7, 8, 10, 12, 17, or 20 by delivering an anti-bacterial/viral agent to suppress the target bacterial/viral species.

When a proposed FMT donor whose stool is tested and found to contain an insufficient level of one or more of the beneficial bacterial or viral species such as Bacteroides dorei or shown in Tables 4-6, 9, 11, 13, 18, 19, and 21 (e.g., each is less than about 0.01% of total bacteria/viruses in the stool sample), the proposed donor is deemed as an unsuitable donor for FMT intended to treat COVID-19 symptoms or to facilitate patient recovery from COVID-19, he may be disqualified as a donor in favor of anther individual whose stool sample exhibits a more favorable bacterial/viral profile, and his fecal material should not be immediately used for FMT due to the lack of prospect of conferring such beneficial health effects unless the stool material is adequately modified. In these cases of expected lack of health benefits from FMT treatment can be readily improved in view of the inventors' discovery, for example, one or more of the bacterial species shown in Tables 4, 5, 9, 13, and 18 or belonging to the bacterial taxa shown in Tables 19 and 21, and/or one or more of the viral species shown in Table 11 may be introduced from an exogenous source into a donor fecal material so that the level of the bacterial or viral species in the fecal material is increased (e.g., to reach at least about 0.1% of total bacteria or viruses in the fecal material) before it is processed for use in FMT for the treatment of COVID-19 symptoms or for COVID-19 patient recovery. Pre-treatment schemes with similarly intended goals can be employed to prepare patients who are soon to receive FMT treatment in order to maximize their potential to receive health benefits such as those stated above and herein.

As an alternative, the beneficial bacterial species/taxa or viral species (one or more of those shown in Tables 4, 5, 9, 13, 18, 19, and 21 or Table 11, respectively) may be obtained from a bacterial or viral culture in a sufficient quantity and then formulated into a suitable composition, which is without any fecal material taken from a donor, for delivery into a COVID-19 patient's gut. Similar to FMT, such composition can be introduced into a patient by oral, nasal, or rectal administration.

On the other hand, certain bacterial species/taxa or viral species (e.g., those in Table 3, 7, 8, 10, 12, 17, or 20) are found to rise in their level or relative abundance in COVID-19 patients with a more severe disease or a higher SARS-CoV-2 virus load. Thus, COVID-19 patients are treated to reduce the level of these bacterial species/taxa or viral species in order to reduce disease severity and facilitate the patients' recovery from the illness. There are several options to reduce the level of these bacterial or viral species: first, the patient may be given a specific anti-bacterial/viral agent to specifically kill or suppress the targeted bacterial/viral species, thereby lowering the abnormally high level of these bacteria or viruses.

Second, the patient may be first given an anti-bacterial/viral agent, such as a broad spectrum antibiotic or antiviral agent to kill or suppress all bacterial or viral species, or a specific anti-bacterial or anti-viral agent to specifically kill or suppress the targeted bacterial or viral species; then a composition may be administered to the patient (e.g., by FMT) to introduce a well-balanced mixed bacterial culture and/or viral culture into the GI tract of the patient.

Third, if the COVID-19 patient has already received antibiotic treatment or generic antiviral therapy, for example, as a part of anti-pneumonia treatment or treatment to suppress coronavirus proliferation, and already has a significantly suppressed bacterial presence and/or viral presence in his GI tract, then a composition containing an appropriate mixed bacteria culture and/or viral culture (e.g., processed fecal matter from a suitable donor) may be directly administered to the patient in order for the bacteria/virus mix to be introduced to the GI tract.

Each of these options can be performed in one combined step to achieve the first and second treatment method goals, i.e., to increase the level of certain bacterial species/taxa or viral species (such as one or more of those shown in Tables 4-6, 9, 11, 13, 18, 19, and 21 and Bacteroides dorei) and to decrease the level of certain other bacterial species (for example, one or more of those listed in Tables 3, 7, 8, 10, 12, 17, and 20), using one single composition (such as processed fecal material from an FMT donor) containing the pertinent bacterial or viral species within the appropriate ratio range to one another.

Immediately upon completion of the step of introducing an effective amount of the desired bacterial and/or viral species into a patient's GI tract (e.g., via an FMT procedure) and/or the step of suppressing undesirable bacteria and/or virus level, the recipient may be further monitored by continuous testing of the level or relative abundance of the bacterial and/or viral species in the stool samples on a daily basis for up to 5 days post-procedure while the clinical symptoms of COVID-19 being treated as well as the general health status (e.g., bodyweight, blood cholesterol, triglyceride, low-density lipoprotein cholesterol (LDL-C) and/or high-density lipoprotein cholesterol (HDL-C) levels) of the patient are also being monitored in order to assess treatment outcome and the corresponding levels of relevant bacteria and/or viruses in the recipient's GI tract: the level of pertinent bacterial or viral species may be monitored in connection with observation of patient improvement and recovery from COVID (e.g., time needed for resolution of clinical symptoms or for patient to reach PCR-negative for SARS-CoV-2) as well as the general health benefits achieved such as weight maintenance, blood glucose level, blood cholesterol level, blood triglyceride level, and blood HDL-C/LDL-C levels.

### IV. Assessing Disease Severity and Corresponding Treatment

The present inventors also discovered that the altered level of certain bacterial or viral species can indicate the severity of COVID-19: they revealed the correlation between reduced level of certain bacterial species/taxa or viral species (e.g., Bacteroides dorei, one or more of those shown in Table 4, 5, 6, 9, 13, 18, 19, 21, or 11, respectively) in the patients' stool sample and the likelihood of a more severe disease outcome, for example, a higher likelihood of longer recovery time, developing pneumonia, the need to be intubated, and up to death. Similarly, a correlation between increased level of certain other bacterial species/taxa or viral species (e.g., one or more of those shown in Table 3, 7, 8, 12, 17, 20, or 10, respectively) and the likelihood of a more severe disease outcome has been established.

Thus, when stool samples taken from two or more COVID-19 patients, the level or relative abundance of Bacteroides dorei or any one of the bacterial species/taxa or viral species in Tables 4-13 and 17-21 in the samples may be determined, for example, by PCR especially quantitative PCR. For the bacterial species/taxa or viral species listed in Table 4, 5, 6, 9, 11, 13, 18, 19, or 21 or Bacteroides dorei, a lower level indicates higher severity or a worse clinical outcome of the disease for the patient; conversely, for the bacterial or viral species listed in Table 3, 7, 8, 10, 12, 17, or 20, a higher level indicates higher severity or a worse clinical outcome of the disease for the patient. In the event that the level of multiple species are measured and compared, the severity determination is made based on the indication from the majority of the pertinent bacterial or viral species measured.

Once the disease severity or clinical outcome assessment is made, for example, patient A is deemed more likely to suffer a more severe form of COVID-19 with worse clinical outcome than patient B, differential treatment steps can be optionally taken as a measure to address the heightened risk for patient A. For example, patient A would be given more aggressive treatment options such as hospitalization and administration of therapeutic agents known to be effective for treating COVID-19 such as antiviral agent ivermectin or hydroxychloroquine with zinc and an antibiotic such as Azithromycin or doxycycline, whereas patient B whose risk for adverse clinical outcome is deemed low may be prescribed home observation without any prescription medication.

### V. Kits and Compositions for Use in COVID-19 Treatment

The present invention also provides novel kits and compositions that can be used for improving therapeutic efficacy and conferring health benefits in the therapeutic and/or prophylactic treatment of COVID-19, including facilitation of patient recovery process. For example, a kit is provided that comprises a first container containing a first composition comprising (i) an effective amount of bacterial species Bacteroides dorei or one or more of the bacterial species set forth in Tables 4, 5, 9, 13, and 18 or belonging to any one of the bacterial taxa set forth in Tables 19 and 21, (ii) an effective amount of one or more of the viral species set forth in Table 11, (iii) an effective amount of an anti-bacterial agent that suppresses growth of one or more of the bacterial species set forth in Tables 3, 8, 12, and 17 or belonging to any one of the bacterial taxa set forth in Table 20, or (iv) an effective amount of an anti-viral agent that suppresses growth of one or more of the viral species set forth in Table 10, and a second container containing a second composition comprising a prebiotic or a therapeutic agent effective for treating COVID-19 (e.g., antiviral agent ivermectin or a combination of hydroxychloroquine with zinc sulfate and Azithromycin or doxycycline).

In some cases, the first composition comprises a fecal material from a donor, which has been processed, formulated, and packaged to be in an appropriate form in accordance with the delivery means in the FMT procedure, which may be by direct deposit in the recipient's lower gastrointestinal track (e.g., wet or semi-wet form) or by oral ingestion (e.g., frozen, dried/lyophilized, encapsulated). Alternatively, the first composition may not contain any donor fecal material but is an artificially mix containing the preferred bacterial and/or viral species, such as bacterial species Bacteroides dorei or one or more of the bacterial species set forth in Tables 4, 5, 9, 13, and 18 or belonging to any one of the bacterial taxa set forth in Tables 19 and 21, or one or more of the viral species set forth in Table 11, at an appropriate ratio and quantity. Further, the first composition may contain an adequate amount of an anti-bacterial agent that suppresses growth of one or more of the bacterial species set forth in Tables 3, 8, 12, and 17 or belonging to any one of the bacterial taxa set forth in Table 20, and/or an effective amount of an anti-viral agent that suppresses growth of one or more of the viral species set forth in Table 10. The anti-bacterial or anti-viral agent may be a broad-spectrum anti-bacterial or anti-viral agent in some cases; or in other cases it may be a specific anti-bacterial or anti-viral agent targeting the specific bacterial species/taxa or viral species only (e.g., those set forth in Table 3, 8, 10, 12, or 17, or belonging to the bacterial taxa set forth in Table 20): it may be a short polynucleotide, e.g., a small inhibitory RNA, microRNA, miniRNA, lncRNA, or an antisense oligonucleotide, that is capable of specifically targeting one or more of predetermined bacterial or viral species without significantly affecting other closely related bacterial or viral species.

In other cases, the first composition may be a composition (e.g., a processed FMT donor fecal material) comprising the preferred bacterial or viral species (such as one or more of the bacterial species/taxa or viral species selected from Bacteroides dorei and those set forth in Tables 4, 5, 9, 13, 18, 19, 21, and 11) at an appropriate ratio and quantity along with a specific anti-bacterial or anti-viral agent targeting the specific bacterial species/taxa or viral species only (e.g., those in Tables 3, 8, 12, 17, 20, and 10). The first composition is formulated and packaged in accordance with its intended means of delivery to the patient, for example, by oral ingestion, nasal delivery, or rectal deposit.

The second composition in some cases may comprises an adequate or effective amount of a prebiotic or a therapeutic agent effective for treating COVID-19, for example, ivermectin, atovaquone, daclatavir, favipiravir, remdesivir, simeprevir, saquinavir, tolicizumab, the combination of lopinavir, ritonavir, and INFβ, and the combination of a zinc ionophore such as hydroxychloroquine or quercetin, a zinc salt, and an antibiotic such as azithromycin or doxycycline. The composition is formulated for the intended delivery method of the prebiotic or therapeutic agent(s), for example, by injection (intravenous, intraperitoneal, intramuscular, or subcutaneous injection) or by oral/nasal administration or by local deposit (e.g., suppositories).

The first and second compositions are often kept separately in two different containers in the kit. In some cases, the composition for increasing the level of certain bacterial or viral species (such as bacterial species Bacteroides dorei or one or more of the bacterial species set forth in Tables 4, 5, 9, 13, and 18 or belonging to any one of the bacterial taxa set forth in Tables 19 and 21, or one or more of the viral species set forth in Table 11) and the composition for suppressing other bacterial species/taxa or viral species (e.g., one or more of the bacterial species set forth in Tables 3, 8, 12, and 17 or belonging to any one of the bacterial taxa set forth in Table 20, or one or more of the viral species set forth in Table 10) may be combined to form a single composition for administration to the patient together, for example, by oral or local delivery, at the same time. In some cases, the first and second compositions may be combined in a single composition so that they can be administered to the patient together, for example, by oral or local delivery, at the same time.

Lastly, a kit is provided for the quantitative detection of one or more bacterial species such as one or more of BD, those set forth in Tables 4-13 and 17-21, or those belonging to the bacterial taxa set forth in Tables 19-21, or for the quantitative detection of one or more of the viral species set forth in Tables 10 and 11. The kit comprises a set of oligonucleotide primers for the amplification, such as PCR, of a polynucleotide sequence derived from, and preferably unique to, any one of the pertinent bacterial species/taxa or viral species (such as one or more of those set forth in Tables 4-13 and 17-21).

### EXAMPLES

The following examples are provided by way of illustration only and not by way of limitation. Those of skill in the art will readily recognize a variety of non-critical parameters that could be changed or modified to yield essentially the same or similar results.

### Example 1

### Background

The current COVID-19 pandemic provides a unique opportunity for studying changes in gut microbiota due to this viral disease and for exploring potential new therapeutic approaches for addressing symptoms and detrimental effects caused by this and other viral infections, including those affecting the respiratory system.

### Methods

Cohort description and study subjects

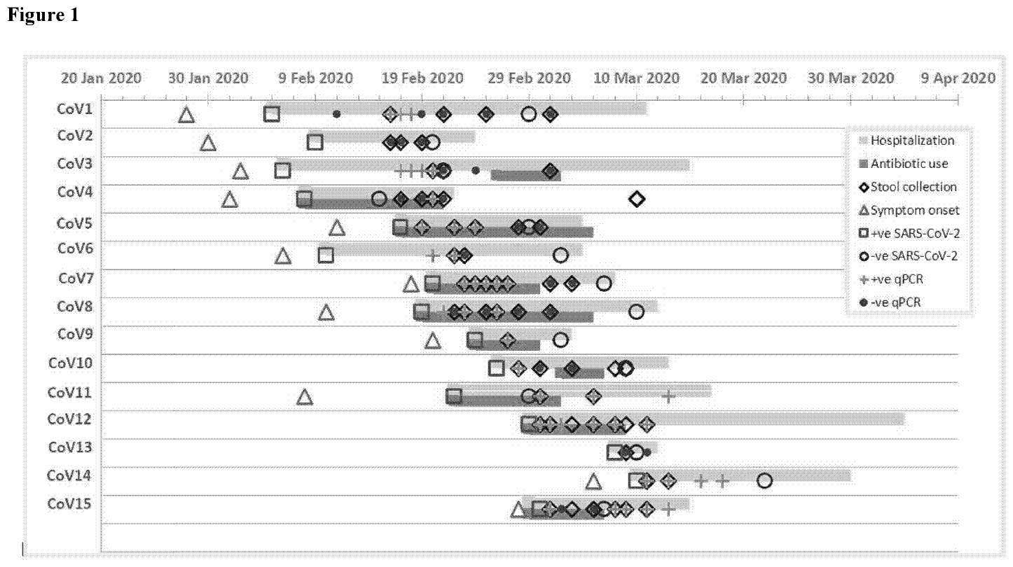

A total of 36 subjects were recruited including 15 patients hospitalized with laboratory-confirmed COVID-19 infection (COVID-19 case), 6 patients hospitalized for pneumonia and tested negative for COVID-19 (pneumonia control) and 15 healthy individuals (healthy control) (Table 1). Clinical characteristics are listed in Table 2. Stool samples from COVID-19 patients were collected serially every 2-3 days until discharge, and one additional stool sample was collected 1 week after discharge. Stool samples from subjects with pneumonia and without COVID-19 (pneumonia control) and healthy individual (healthy control) were collected once at recruitment (FIG. 1).

All patients with laboratory-confirmed COVID-19 hospitalized at the Prince of Wales Hospital and the United Christian Hospital, Hong Kong, from 5 February to 17 March 2020. SARS-CoV-2 infection was confirmed by two RT-PCR targeting different regions of the RdRp gene performed by the local hospital and Public Health Laboratory Service. All participants were followed until hospital discharge or 4 Apr. 2020. Disease severity was categorized as (i) mild, if there was no radiographic evidence of pneumonia; (ii) moderate, if pneumonia was present; (iii) severe, if respiratory rate≥30/min, or oxygen saturation≤93% when breathing ambient air; or (iv) critical, if there was respiratory failure requiring mechanical ventilation, shock, or organ failure requiring intensive care¹. The patients with pneumonia without COVID-19 infection were hospitalized at medical wards and intensive care units at the Prince of Wales Hospital.

TABLE 1
Subjects characteristics

| | COVID-19 | Pneumonia | Healthy |
|---|---|---|---|
| Variables | cases | cases | controls |
| Number | 15 | 6 | 16 |
| Male | 7 (47%) | 4 (67%) | 9 (56%) |
| Age, years | 55 (44, 67.5) | 50 (44, 65) | 48 (45, 48) |
| Comorbidities | 6 (40%) | 6 (100%) | 3 (19%) |
| Recent exposure history | | | |
| Travel to Wuhan City | 0 (0%) | 0 (0%) | |
| Travel to other cities of | 1 (7%) | 0 (0%) | |
| Hubei provincial | | | |
| Contact with person | 5 (33%) | 0 (0%) | |
| with COVID19 | | | |
| Have family cluster | 4 (27%) | 0 (0%) | |
| outbreak | | | |
| Symptoms at admission | | | |
| Fever | 9 (60%) | 4 (67%) | |
| Gastrointestinal | | | |
| symptoms | | | |
| Diarrhea | 1 (7%) | 2 (33%) | |
| Respiratory symptoms | | | |
| Cough | 11 (73%) | 4 (67%) | |
| Sputum | 5 (33%) | 3 (50%) | |
| Sore throat | 0 (0%) | 0 (0%) | |
| Rhinorrhea | 3 (20%) | 1 (17%) | |
| Shortness of Breath | 4 (27%) | 3 (50%) | |
| Blood result | | | |
| Lymphocytes (×10⁹/L, | 0.9 (0.7, 1.1) | 1.1 (0.9, 1.2) | |
| normal range 1.1-2.9) | | | |
| Antibiotics therapy | 10 (67%) | 6 (100%) | |
| 1 type of antibiotics | 4 (27%) | 2 (33%) | |
| 2 types of antibiotics | 5 (33%) | 2 (33%) | |
| 3 types of antibiotics | 1 (7%) | 2 (33%) | |
| Antiviral therapy | 13 (87%) | 0 (0%) | |
| Kaletra | 13 (87%) | 0 (0%) | |
| Ribavirin | 7 (47%) | 0 (0%) | |
| Interferon beta-1b | 1 (7%) | 0 (0%) | |
| Hospitalization | | | |
| Discharge from hospital | 14 (93%) | 4 (67%) | |
| Death | 0 (0%) | 0 (0%) | |

Values are expressed in number (percentage) and median (interquartile range).

TABLE 2
Clinical characteristics of each subject

| | | | | | Symptoms at | | Ad- | | | Blood | |
|---|---|---|---|---|---|---|---|---|---|---|---|
| | | | | Recent | admission | | mitted | | | routine | |
| | | | | exposure | Fever and | | to | Medication | | Lympho- | Chest X-ray |
| Case | Sex | Age | Co-morbidities | history | respiratory | GI | ICU | Antibiotics | Antiviral | cytes* | findings |
| CoV1 | F | 65 | Hypothyroidism, | No | Fever, | nil | Yes | nil | Kaletra, | 1 | Bilateral LZ |
| | | | hypertension, | | cough, | | | | ribavirin | | haziness |
| | | | Chronic hepatitis B | | sputum | | | | | | |
| | | | carrier | | | | | | | | |
| CoV2 | F | 55 | None | Contact | Fever, runny | nil | No | nil | Kaletra | 1.2 | Bilateral LZ |
| | | | | with | nose | | | | | | haziness |
| | | | | person | | | | | | | |
| | | | | with | | | | | | | |
| | | | | COVID19 | | | | | | | |
| CoV3 | M | 42 | None | Travel to | Fever, cough | nil | Yes | Daptomycin | nil | 0.6 | Worsening |
| | | | | Hubei | | | | | | | RLZ haziness, |
| | | | | province | | | | | | | RLL collapse |
| | | | | | | | | | | | re-opened |
| CoV4 | M | 70 | Hyperlipidemia, | No | Sputum, | nil | No | Augmentin, | Kaletra | 0.6 | Bilateral lung |
| | | | duodenal ulcer | | shortness of | | | doxycyline | | | haziness |
| | | | | | breath | | | | | | |
| CoV5 | M | 58 | None | No | Fever, cough | Diarrhea | No | Ceftrixaxone, | Kaletra, | 0.9 | Slight RLZ |
| | | | | | | | | augmentin, | ribavirin | | haziness |
| | | | | | | | | doxycycline | | | |
| CoV6 | M | 71 | None | No | Fever, | nil | No | nil | Kaletra | 1 | Bilateral lung |
| | | | | | cough, | | | | | | infiltration |
| | | | | | shortness of | | | | | | |
| | | | | | breath | | | | | | |
| CoV7 | M | 48 | Diabetes, | No | Fever, cough | nil | No | Augmentin | Kaletra, | N/A | LLZ haziness |
| | | | hypertension, | | | | | | ribavirin | | |
| | | | hyperlipidemia | | | | | | | | |
| CoV8 | F | 38 | None | No | Fever, | nil | No | Ceftrixaxone, | Kaletra | 0.7 | Bilateral LZ |
| | | | | | cough, | | | doxycycline | | | infiltrates |
| | | | | | sputum, | | | | | | |
| | | | | | runny nose | | | | | | |
| CoV9 | M | 33 | None | Contact | Fever, cough | nil | No | Doxyxyxline | Kaletra, | 0.7 | Bilateral LZ |
| | | | | with | | | | | ribavirin | | haziness |
| | | | | person | | | | | | | |
| | | | | with | | | | | | | |
| | | | | COVID19 | | | | | | | |
| CoV10 | F | 70 | Obesity, | No | Cough | nil | No | Ceftrixaxone, | Kaletra, | 0.8 | Bilateral LZ |
| | | | hypertension, | | | | | piperacillin + | ribavirin | | haziness |
| | | | | | | | | tazobactam | | | |
| CoV11 | M | 62 | Diabetes, | No | Fever, | nil | No | Doxycycline, | Kaletra | N/A | Bilateral lung |
| | | | hyperlipidemia, | | cough, | | | sulperazon | | | infiltrates |
| | | | left subclavian | | sputum, | | | | | | |
| | | | artery occlusion | | shortness of | | | | | | |
| | | | | | breath | | | | | | |
| CoV12 | F | 71 | Hypertension, | Contact | Cough | nil | N/A | Ceftrixaxone, | Kaletra, | N/A | N/A |
| | | | renal impairment, | with | | | | azithromycin | ribavirin | | |
| | | | hyperlipidemia | person | | | | | | | |
| | | | | with | | | | | | | |
| | | | | COVID19 | | | | | | | |
| CoV13 | F | 47 | None | Contact | nil | nil | No | nil | nil | 1.9 | No definite |
| | | | | with | | | | | | | consolidation |
| | | | | person | | | | | | | |
| | | | | with | | | | | | | |
| | | | | COVID19 | | | | | | | |
| CoV14 | F | 22 | None | Contact | Fever, runny | nil | No | nil | nil | 1.8 | N/A |
| | | | | with | nose | | | | | | |
| | | | | person | | | | | | | |
| | | | | with | | | | | | | |
| | | | | COVID19 | | | | | | | |
| CoV15 | F | 46 | None | Contact | Cough, | nil | No | Augmentin | Kaletra, | N/A | Clear |
| | | | | with | shortness of | | | | ribavirin, | | |
| | | | | person | breath | | | | interferon | | |
| | | | | with | | | | | beta-1b | | |
| | | | | COVID19 | | | | | | | |
| P1 | F | 69 | Hypertension, | No | Fever | nil | No | Augmentin, | nil | 0.6 | LMZ |
| | | | diabetes, Gastric | | | | | azithromycin, | | | pneumonia |
| | | | antral vascular | | | | | piperacillin + | | | |
| | | | ectasia, tricuspid | | | | | tazobactam | | | |
| | | | regurgitation | | | | | | | | |
| P2 | M | 43 | Fatty liver | No | Cough | nil | No | Piperacillin + | nil | 2.4 | Rt sided |
| | | | | | | | | Tazobactam | | | haziness |
| P3 | F | 92 | Diabetes, | No | Cough, | nil | No | Ceftrixaxonel, | nil | 1.2 | bilateral lung |
| | | | Hypertension,, | | sputum, | | | Azithromycin | | | infiltrate |
| | | | pulmonary | | shortness of | | | | | | |
| | | | fibrosis, | | breath | | | | | | |
| | | | Paroxysmal atrial | | | | | | | | |
| | | | fibrillation, Acute | | | | | | | | |
| | | | coronary | | | | | | | | |
| | | | syndrome | | | | | | | | |
| P4 | M | 47 | Diabetes | No | Fever, | nil | No | Augmentin, | nil | 1 | left effusion, |
| | | | | | sputum | | | Cefotaxime | | | I LMZ |
| | | | | | | | | sodium | | | haziness |
| P5 | M | 36 | Ischemic priapism | No | Fever, | Diarrhea | No | Ceftrixaxonel, | nil | 1.1 | N/A |
| | | | | | cough, | | | Augmentin, | | | |
| | | | | | sputum, | | | Cefotaxime | | | |
| | | | | | shortness of | | | sodium | | | |
| | | | | | breath | | | | | | |
| P6 | M | 52 | Epilepsy, Hepatitis | No | Fever, | Diarrhea | No | Ceftrixaxonel | nil | 0.9 | N/A |
| | | | | | cough, runny | | | | | | |
| | | | | | nose, | | | | | | |
| | | | | | shortness of | | | | | | |
| | | | | | breath | | | | | | |

*Value in ×10⁹/L, normal range 1.1-2.9
LZ: lower zone

### Fecal DNA Extraction

Approximately 100 mg from each stool sample was prewashed with 1 ml ddH2O and pelleted by centrifugation at 13,000×g for 1 min. The pellet was resuspended in 800 μl TE buffer (pH 7.5), supplemented with 1.6 μl 2-mercaptoethanol and 500 U lyticase (Sigma), and incubated at 37° C. for 60 min. The sample was then centrifuged at 13,000×g for 3 min and the supernatant was discarded. After this pretreatment, DNA was subsequently extracted from the pellet using a Maxwel® RSC PureFood GMO and Authentication Kit (Promega) following manufacturer's instructions.

### Metagenomic Sequencing and Analysis

After quality control procedure by using qubit 2.0, qualified DNA is cut into fragments by restriction enzyme. Then the construction of the DNA libraries was completed through the processes of end repairing, adding A to tails, purification and PCR amplification by using Nextera DNA Flex Library Preparation kit (Illumina). The qualified libraries from extracted fecal DAN were then sequenced were sequenced (150 bp paired-end) by on the Illumina NextSeq 550.

Raw sequence reads were filtered and quality-trimmed using Trimmomatic v0.36 1 as follows: 1. Trimming low quality base (quality score <20), 2. Removing reads shorter than 50 bp, 3. Tracing and cutting off sequencing adapters. Contaminating human reads were filtering using Kneaddata (website: bitbucket.org/biobakery/kneaddata/wiki/Home, Reference database: GRCh38 p12) with default parameters. Profiling of bacterial taxonomy from metagenomes of fecal DNA was extracted using MetaPhlAn2 (V2.9) by mapping reads to clade-specific markers. The significantly differential bacteria taxa between COVID-19 group, pneumonia control and healthy control were identified by Multivariate Association with Linear Models (MaAsLin, website: huttenhower.sph.harvard.edu/galaxy/).

### Analysis of the Bacterial Microbiome

Profiling of the composition of bacterial communities was performed on metagenomic trimmed reads via MetaPhlAn2 (v2.7.5)¹. Mapping reads to clade-specific markers gene and annotation of species pangenomes was done through Bowtie2 (v2.3.4.3)². The output table contained bacterial species and its relative abundance in different levels, from kingdom to strain level.

### Statistical Analysis

The significantly differential bacteria taxa between COVID-19 group (with and without antibiotic treatment at baseline), pneumonia controls and healthy controls were identified by Multivariate Association with Linear Models (MaAsLin, website: huttenhower.sph.harvard.edu/galaxy/). Identification of Bacterial species correlated fecal viral load or disease severity were performed via Lasso (least absolute shrinkage and selection operator) analysis.

### Results and Findings

### Section I

### Fecal Microbiome Diversity and Richness of COVID-19 Patients

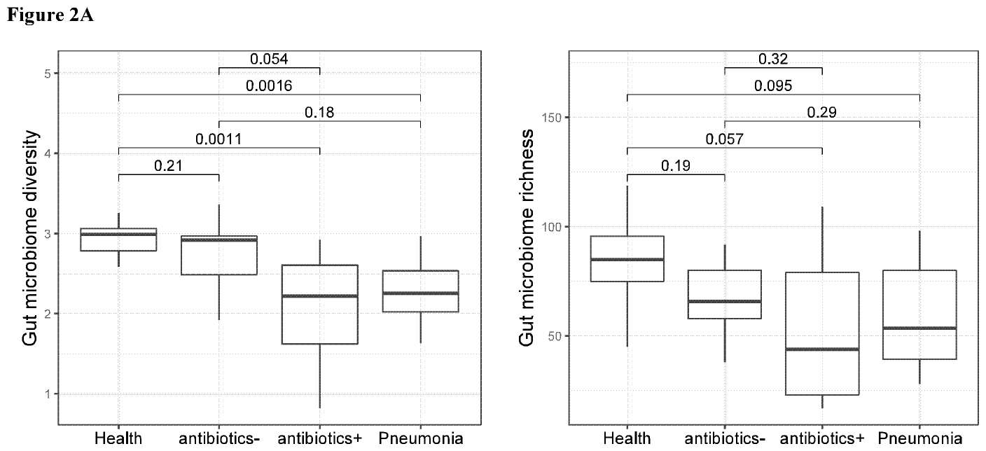

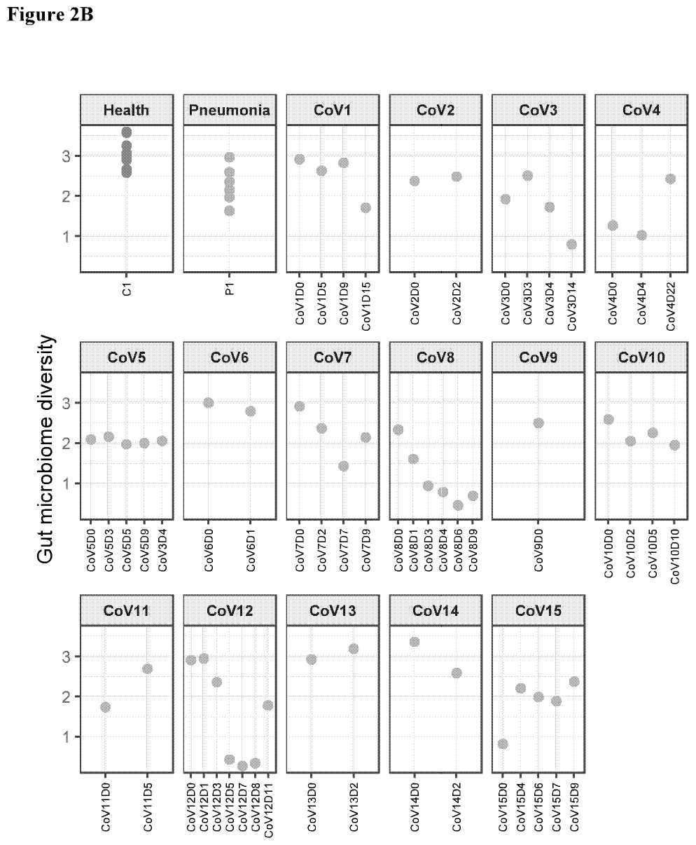

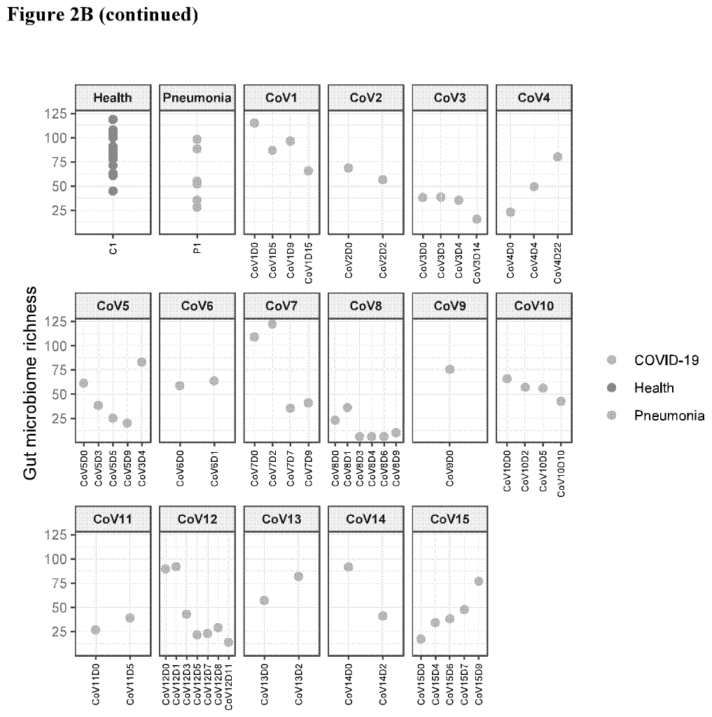

The fecal microbiome diversity and richness of COVID-19(Abx-) were slightly lower than healthy controls. Antibiotics treatment on COVID-19 patients, as compared to non-antibiotics treatment, further decreased the diversity and richness of the fecal microbiome, to a similar level to that of the Pneumonia control patients (FIG. 2a). ICU warded COVID-19 patients, CoV1 and CoV3, had continuously decreased microbiome diversity and richness over the course of hospitalization (FIG. 2b). The microbiome diversity and richness of COVID-19 patients got increased before discharge, shown in patients CoV2, 4, 11, 13, 15 (FIG. 2b). These data indicate that a gradually recovered gut microbiome in COVID-19 patient during clearance of SARS-CoV-2 virus.

### Different Gut Bacterial Profile in COVID-19 Case, Pneumonia Control, and Healthy Control

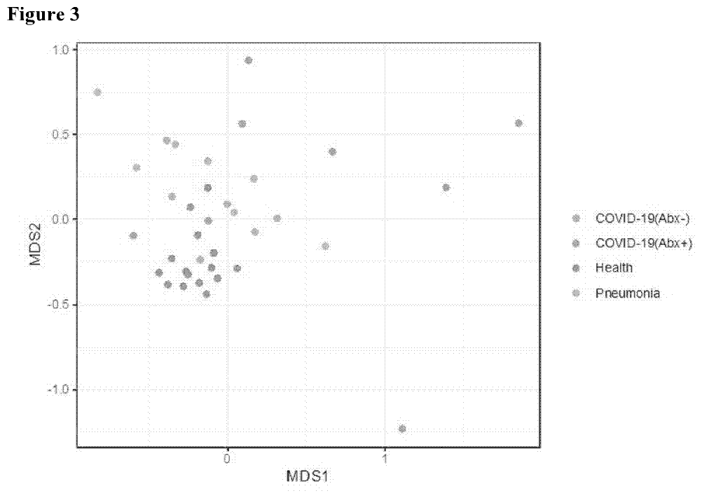

At the bacterial community structure level, healthy subjects' microbiomes clustered together and were more homogenous, whereas the microbiomes of antibiotics naïve COVID-19 patients [COVID-19 (Abx-)] clustered away from healthy microbiomes, indicating there was a dysbiosis in COVID-19 (FIG. 3). Antibiotics regimen further shifted the COVID-19 microbiome away from the healthy microbiome.

COVID-19 patients and pneumonia control patients partly overlapped with each other in their microbiome clusters, indicating that COVID-19 and pneumonia had a shared microbiome feature whilst each had its own microbiome feature (FIG. 3).

The compositional differences among the microbiome of healthy controls, COVID-19 (Abx-), COVID-19 (Abx+), and Pneumonia control patients were then investigated. Eubacterium ventriosum, an anti-inflammatory bacterium, was universally underrepresent across COVID-19 (Abx-), COVID-19 (Abx+), and Pneumonia control patients (Table 4). COVID-19 (Abx-) patients were specifically enriched for the pathogenic bacteria, Actinomyces viscosus, Clostridium hathewayi and Bacteroides nordii (Table 3). In contrast, COVID-19 (Abx+) and Pneumonia control patients had a depletion of a series of symbiotic bacteria, including a short-chain fatty acid producer Lachnospiraceae bacterium_5_1_63FAA (Table 5).

In addition, COVID-19 (Abx+) had specific underrepresentation of Dorea formicigenerans, Fecalibacterium prausnitzii, Eubacterium rectale, and Ruminococcus obeum (all are symbionts beneficial to host health) (Table 5); Pneumonia control patients had specific underrepresentation of Enterococcus faecium and Clostridium ramosum (both are opportunistic pathogens).

These data indicate that SARS-CoV-2 infection can cause disease-specific gut microbiome alteration, where pathogens tend to enrich while a diversity of salutary symbionts were lost.

Surprisingly, all the underrepresented salutary symbionts (listed in Tables 4 and 5) maintained absent or a very low abundance in COVID-19 patients during hospitalization, even when SARS-CoV-2 virus was cleared and pneumonia symptoms dismissed. This indicates that the loss of salutary microbes in the gut of COVID-19 patients may be irreversible (or get recovered very slowly, if there is), which warrants further nutritional or probiotic supplementation on COVID-19 patients to improve their gut microbiome diversity and health.

TABLE 3
Bacterial species specifically enriched in COVID-19 Abx−

| Bacterial Species | NCBI:txid |
|---|---|
| Actinomyces viscosus | 1656 |
| Clostridium hathewayi | 154046 |
| (preferable strain: Hungatella hathewayi | (999412) |
| 12489931) | |
| Bacteroides nordii | 291645 |

TABLE 4
Bacterial species underrepresented in
both COVID-19 and pneumonia

| Bacterial Species | NCBI:txid |
|---|---|
| Eubacterium ventriosum | 39496 |

TABLE 5
Bacterial taxa underrepresented in COVID-19 Abx+

| Bacterial Taxa | NCBI:txid |
|---|---|
| Dorea formicigenerans (species) | 39486 |
| Blautia (genus) | 572511 |
| Faecalibacterium (genus) | 216851 |
| Faecalibacterium prausnitzii (species) | 853 |
| Eubacteriaceae (family) | 186806 |
| Eubacterium (genus) | 1730 |
| Eubacterium rectale (species) | 39491 |
| Ruminococcaceae (genus) | 541000 |
| Roseburia (genus) | 841 |
| Coprococcus (genus) | 33042 |
| Ruminococcus obeum (species) | 40520 |
| Lachnospiraceae bacterium | 658089 |
| 5_1_63FAA (species) | |

These observations indicate that therapeutic benefits can be gained in the treatment of COVID-19 infection by reducing the relative abundance of bacteria listed in Table 3 in a patient's gastrointestinal tract. One method is by performing intestinal microbiota transplantation.

Furthermore, bacteria listed in Table 4 and Table 5 can be administered to patients with COVED-19, either individually or in combination, for therapeutic benefits in the treatment of COVID-19. This can also be combined with other treatment as an adjunct therapy.

Lastly, bacteria listed in Table 4 and Table 5 can be administered to patients recovered from COVID-19 as a nutritional or probiotic supplementation on COVID-19 patients to improve their gut microbiome diversity and health.

Positive Correlation between Fecal Viral Road of SARS-CoV-2 and Rothia mucilaginosa

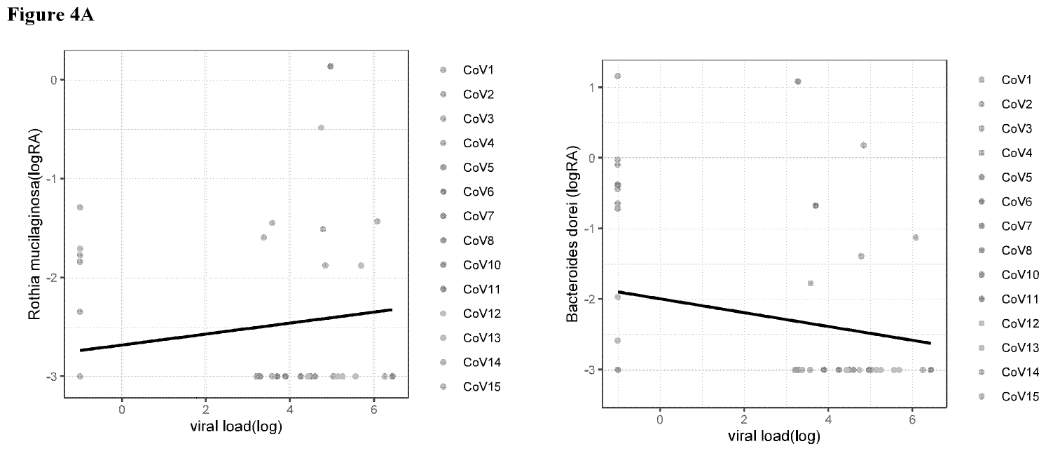

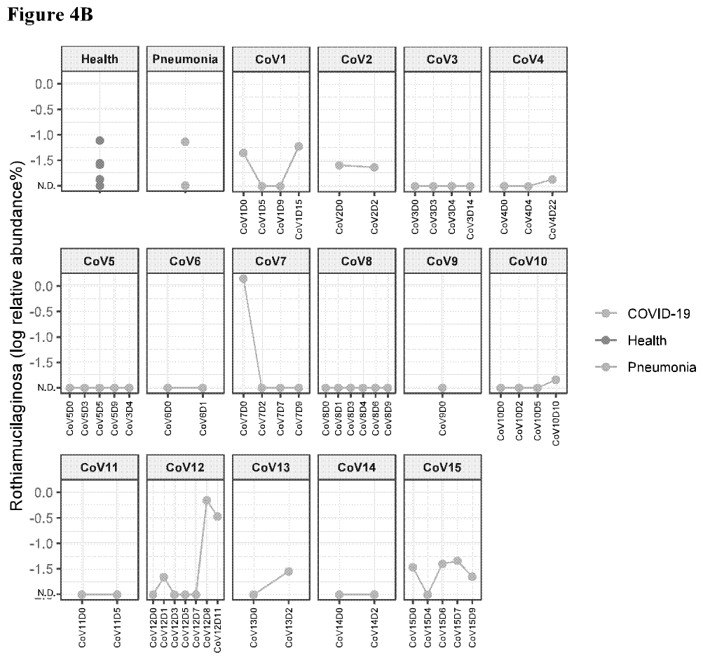

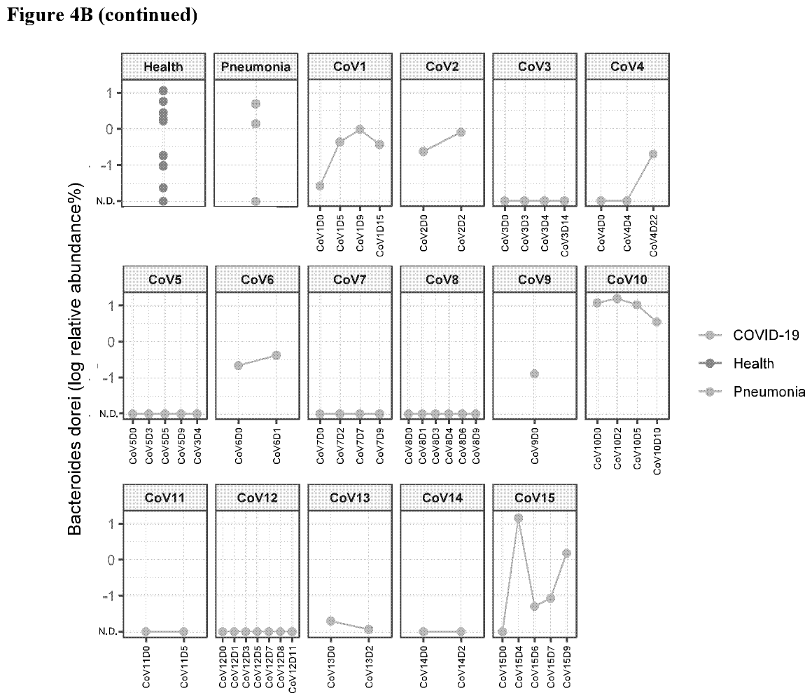

The fecal viral road of SARS-CoV-2 showed a positive correlation with the bacterium Rothia mucilaginosa (FIG. 4). Rothia mucilaginosa is part of normal microflora of the human mouth and the upper respiratory tract, and an opportunistic pathogen affecting immunocompromised hosts resulting in bacterial pneumonia. Data from this study showed that Rothia mucilaginosa showed a very high abundance in the gut of a subset of COVID-19 patients who also had very high SARS-CoV-2 loads (CoV7, 12, 15). While SARS-CoV-2 virus was cleared from patient CoV7, Rothia mucilaginosa was disappeared as well in the feces. However, it persisted in the feces of patients CoV12 and CoV15 along with the prolonged high fecal shedding of SARS-CoV-2 virus.

Interestingly, the fecal viral road of SARS-CoV-2 showed an inverse correlation with the bacterium Bacteroides dorei (NCBI:txid 483217, FIG. 4), an anti-inflammatory bacterium that is underpresent in IBD. At admission, patients who had a very high fecal SARS-CoV-2 load showed an absence or remarkable lack of Bacteroides dorei (patients CoV1, 3, 5, 6, 7, 11, 12, 14, 15), compared to healthy subjects. Patients who showed clearance or decrease of fecal SARS-CoV-2 virus during hospitalization (CoV1, 3, 4, 6) experienced an increase in Bacteroides dorei over time. In patient 15, the abundance of Bacteroides dorei and SARS-CoV-2 virus co-varied in an opposite direction during hospitalization. These data indicate Bacteroides dorei may combat SARS-CoV2.

These results indicate that Bacteroides dorei can be administered to patients with COVID-19 infection for treating COVID-19 and associated symptoms, especially for the purpose of facilitating patient recovery from the disease.

TABLE 6
Bacteria negatively correlated with disease severity

| Bacterial Species | NCBI:txid |
|---|---|
| Bifidobacterium (genus) | 1678 |
| Bacteroidesplebeius (species) | 310297 |
| Bacteroidales noname (family) | |
| Bacteroidales bacterium ph8 (species) | 2585118 |
| Parabacteroides merdae (species) | 46503 |

TABLE 7
Bacteria positively correlated with disease severity

| Bacterial Species | NCBI:txid |
|---|---|
| Atopobium rimae (species) | 1383 |
| Parabacteroides unclassified (species) | |
| Firmicutes (phyla) | 1239 |
| Bacillales (order) | 1385 |
| Bacillales noname (family) | |
| Gemella (genus) | 1378 |
| Enterococcaceae (family) | 81852 |
| Enterococcus (genus) | 1350 |
| Streptococcus gordonii (species) | 1302 |
| Clostridium hathewayi (species) | 154046 |

These observations indicate that the bacteria listed in Table 6 and Table 7 can be used, either individually or in different combinations, to predict the severity and outcome of COVID-19. For example, the relative abundance can be determined using as a panel of qPCR primer or by metagenomics sequencing to calculate the predicted severity.

### Section II

### Baseline Gut Microbiome and Disease Severity of COVID-19

To understand whether baseline gut microbiome impacts the severity of COVID-19, association between baseline fecal microbiome and COVID-19 severity (mild, moderate, severe, or critical) was assessed in seven antibiotic-naïve COVID-19 cases. A total of 23 bacterial taxa were found to be significantly associated with COVID-19 disease severity, most of which (15 out of 23) were from the Firmicutes phylum (Table 8 and Table 9). Among them, 8 and 7 Firmicutes members, respectively, showed positive and negative correlation with disease severity. The finding of the association of gut Firmicutes bacteria with COVID-19 severity highlights the importance of bacterial membership in modulating human response to SARS-CoV-2 infection.

Three bacterial members from the Firmicutes phylum, the genus Coprobacillus, the species Clostridium ramosum and Clostridium hathewayi, were the top bacteria positively associated with COVID-19 disease severity (Spearman correlation coefficient Rho>0.9, p<0.01, Table 8). In contrast, two beneficial species Alistipes onderdonkii and Faecalibacterium prausnitzii were top bacteria species to show a negative correlation with COVID-19 severity (Table 9).

In addition, multiple DNA virus species (phages) showed significant positive correlations with COVID-19 severity (Table 10), where Streptococcus phages exhibited the most prominent positive correlation with disease severity (Spearman correlation coefficient Rho=0.69 and 0.64, p=0.001 and 0.003, respectively for Streptococcus virus 2972 and Streptococcus phage phiARI0468-1). In contrast, a multitude of phages of Escherichia and Enterobacteria were inversely correlated with COVID-19 severity (Table 11).

The bacteria listed in Table 8 and Table 9 and the viruses listed in Table 10 and Table 11 can be used individually or in combination to predict the severity and outcome of COVID-19. For example, the relative abundance can be determined using as a panel of qPCR primer or by metagenomics sequencing to calculate the predicted severity.

Also, the bacteria listed in Table 9 and viruses listed in Table 11 can be administered to patients with COVID-19 as a single bacterium or virus or in combination for treating COVID-19. They can also be combined with other treatment as an adjunct therapy.

Further, the bacteria listed in Table 9 and viruses listed in Table 11 can be administered to patients who are recovering or have recovered from COVID-19 as a nutritional or probiotic supplementation for the patients to improve their gut microbiome diversity and health.

TABLE 8
Bacterial taxa positively correlated with COVID-19 severity

| | | | Correlation | |
|---|---|---|---|---|
| | | | coefficient | |
| Bacterial taxa | Level | NCBI:txid | Rho | p value |
| Coprobacillus | genus | 100883 | 0.92 | 0.003 |
| Clostridium ramosum | species | 1547 | 0.92 | 0.003 |
| Clostridium hathewayi | species | 154046 | 0.9 | 0.005 |
| Erysipelotrichia | class | 526524 | 0.9 | 0.006 |
| Erysipelotrichales | order | 526525 | 0.9 | 0.006 |
| Erysipelotrichaceae | family | 128827 | 0.9 | 0.006 |
| Erysipelotrichaceae noname | genus | | 0.9 | 0.006 |
| Actinomyces odontolyticus | species | 1660 | 0.87 | 0.011 |
| Erysipelotrichaceae | species | 469614 | 0.87 | 0.011 |
| bacterium 6_1_45 | | | | |
| Enterobacter | genus | 547 | 0.87 | 0.011 |
| Enterobacter cloacae | species | 550 | 0.87 | 0.011 |
| Parabacteroides unclassified | species | | 0.81 | 0.029 |
| Alistipes indistinctus | species | 626932 | 0.81 | 0.029 |

TABLE 9
Bacterial taxa negatively correlated with COVID-19 severity

| | | | Correlation | |
|---|---|---|---|---|
| | | | coefficient | |
| Bacterial taxa | Level | NCBI:txid | Rho | p value |
| Alistipes onderdonkii | species | 328813 | −0.9 | 0.005 |
| Anaerostipes hadrus | species | 649756 | −0.87 | 0.011 |
| Lachnospiraceae bacterium | species | 658089 | −0.87 | 0.011 |
| 5_1_63FAA | | | | |
| Roseburia | genus | 841 | −0.87 | 0.011 |
| Faecalibacterium | genus | 216851 | −0.87 | 0.011 |
| Faecalibacterium prausnitzii | species | 853 | −0.87 | 0.011 |
| Bacteroides ovatus | species | 28116 | −0.84 | 0.019 |
| Bifidobacterium | species | 28026 | −0.81 | 0.026 |
| pseudocatenulatum | | | | |
| Dorea | genus | 189330 | −0.81 | 0.026 |
| Dorea longicatena | species | 88431 | −0.81 | 0.026 |

TABLE 10
Viral taxa positively correlated with COVID-19 severity

| | | | Correlation | |
|---|---|---|---|---|
| | | | coefficient | |
| Viral taxa | Level | NCBI:txid | Rho | p |
| Herelleviridae | family | 2560065 | 0.43452227 | 0.072 |
| Sphaerolipoviridae | family | 1714267 | 0.41639028 | 0.086 |
| Streptococcus virus 2972 | species | 306323 | 0.69098919 | 0.001 |
| Streptococcus phage | species | 1701827 | 0.64607013 | 0.004 |
| phiARI0468-1 | | | | |
| Enterococcus phage | species | 1445858 | 0.61269814 | 0.007 |
| IME-EFm1 | | | | |
| Brochothrix virus A9 | species | 2560358 | 0.49892554 | 0.035 |
| Pseudomonas virus H66 | species | 1273707 | 0.4856584 | 0.041 |

TABLE 11
Viral taxa negatively correlated with COVID-19 severity

| | | | Correlation | |
|---|---|---|---|---|
| | | | coefficient | |
| Viral taxa | Level | NCBI:txid | Rho | p |
| Inoviridae | family | 10860 | −0.5976137 | 0.009 |
| Myoviridae | family | 10662 | −0.4100128 | 0.091 |
| Pseudomonas phage OBP | species | 1124849 | −0.4764362 | 0.046 |
| Corynebacterium virus Darwin | species | 2560394 | −0.4868065 | 0.040 |
| Ralstonia virus RSL1 | species | 1980923 | −0.4880702 | 0.040 |
| Enterobacteria phage YYZ-2008 | species | 564886 | −0.5010539 | 0.034 |
| Escherichia virus TL2011 | species | 1981169 | −0.5054986 | 0.032 |
| Enterobacteria phage phi80 | species | 10713 | −0.5132551 | 0.029 |
| Enterobacteria phage phiP27 | species | 103807 | −0.517706 | 0.028 |
| Stx2-converting phage 1717 | species | 563769 | −0.5344209 | 0.022 |
| Enterobacteria phage VT2phi 272 | species | 936054 | −0.5369763 | 0.022 |
| Escherichia virus If1 | species | 1977411 | −0.5537297 | 0.017 |

### Fecal SARS-CoV-2 Virus Load and Gut Bacterial Abundance

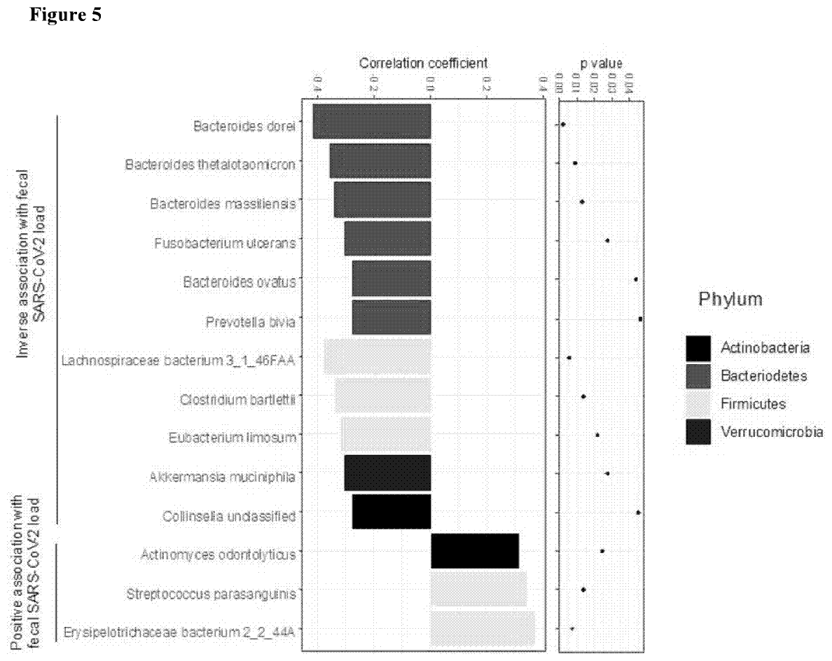

It was investigated whether gut bacteria were associated with fecal SARS-CoV-2 load. A total of 20 bacterial species were identified to be significantly associated with fecal viral load of SARS-CoV-2 across all fecal samples (14 of these species with p<0.05 are shown in FIG. 5). Among them, 6 species were from the Bacteroidetes phylum. Four Bacteroides species, including Bacteroides dorei, Bacteroides thetaiotaomicron, Bacteroides massiliensis, and Bacteroides ovatus, showed significant inverse correlation with fecal SARS-CoV-2 load (all Spearman correlation coefficient Rho<−0.2, p<0.05, FIG. 5). Taken together, these data indicate that Bacteroides species may have a potential protective role in combating SARS-CoV-2 infection by hampering host entry through ACE2. In contrast, Erysipelotrichaceae bacterium 2_2_44A, a Firmicutes species, showed the strongest positive correlation with fecal SARS-CoV-2 load (Spearman correlation coefficient Rho=0.89, p=0.006, FIG. 5). Considering the strong association of baseline abundance of Erysipelotrichaceae with COVID-19 severity (Spearman correlation Rho=0.89,p=0.006, Table 5), gut Erysipelotrichaceae is indicated to play a role in augmenting SARS-CoV-2 infection in the host gut.

TABLE 12
Bacteria species positively correlated with
viral road of SARS-CoV-2

| Phylum | Species | NCBI:txid | Rho | p |
|---|---|---|---|---|
| Firmicutes | Erysipelotrichaceae | 457422 | 0.368 | 0.007 |
| | bacterium 2_2_44A | | | |

TABLE 13
Bacteria species negatively correlated with viral road of SARS-CoV-2

| Phylum | Species | NCBLtxid | Rho | p |
|---|---|---|---|---|
| Bacteriodetes | Bacteroides dorei | 357276 | −0.418 | 0.002 |
| Firmicutes | Lachnospiraceae | 665950 | −0.379 | 0.006 |
| | bacterium 3_1_46FAA | | | |
| Bacteriodetes | Bacteroides thetaiotaomicron | 818 | −0.359 | 0.009 |
| Bacteriodetes | Bacteroides massiliensis | 204516 | −0.342 | 0.013 |
| Firmicutes | Streptococcus parasanguinis | 1318 | 0.340 | 0.014 |
| Firmicutes | Clostridium bartlettii | 261299 | −0.340 | 0.014 |
| Firmicutes | Eubacterium limosum | 1736 | −0.318 | 0.022 |
| Actinobacteria | Actinomyces odontolyticus | 1660 | 0.312 | 0.024 |
| Verrucomicrobia | Akkermansia muciniphila | 239935 | −0.306 | 0.027 |
| Bacteriodetes | Fusobacterium ulcerans | 861 | −0.306 | 0.027 |
| Bacteriodetes | Bacteroides ovatus | 28116 | −0.281 | 0.044 |
| Actinobacteria | Collinsella unclassified | | −0.279 | 0.045 |
| Bacteriodetes | Prevotella bivia | 28125 | −0.278 | 0.046 |
| Bacteriodetes | Bacteroides xylanisolvens | 371601 | −0.264 | 0.059 |
| Bacteriodetes | Bacteroides salyersiae | 291644 | −0.264 | 0.059 |
| Bacteriodetes | Bacteroides stercoris | 46506 | −0.260 | 0.063 |
| Firmicutes | Ruminococcaceae bacterium_D16 | 552398 | −0.250 | 0.074 |
| Firmicutes | Clostridium nexile | 29361 | −0.248 | 0.076 |
| Firmicutes | Lachnospiraceae | 658089 | −0.244 | 0.081 |
| | bacterium_5_l_63FAA | | | |

### REFERENCES

1. Truong D T, Franzosa E A, Tickle T L, et al. MetaPhlAn2 for enhanced metagenomic taxonomic profiling. Nat Methods 2015; 12(10): 902-3.
2. Langmead B, Salzberg S L. Fast gapped-read alignment with Bowtie 2. Nat Methods 2012; 9(4): 357-9.
3. Hadley W, Mara A, Jennifer B, et al. Welcome to the Tidyverse. Journal of Open Source Software 2019; 4(43): 1686.
4. McMurdie P J, Holmes S. phyloseq: an R package for reproducible interactive analysis and graphics of microbiome census data. PLoS One 2013; 8(4): e61217.
5. Segata N, Izard J, Waldron L, et al. Metagenomic biomarker discovery and explanation. Genome Biol 2011; 12(6): R60.

### Example 2

### Background

Although COVID-19 is primarily a respiratory illness, several lines of evidence suggest involvement of the gut microbiome in this disease: (i) meta-analyses have highlighted gastrointestinal (GI) symptoms such as diarrhea, vomiting, and abdominal pains in COVID-19 patients (Cheung et al., 2020a; Vetter et al., 2020); (ii) SARS-CoV-2 can infect and replicate in human small intestine enterocytes (Lamers et al., 2020); and (iii) SARS-CoV-2 RNA is detectable in stools of COVID-19 patients (Wolfel et al., 2020; Xu et al., 2020), indicating in vivo replication in the GI tract. Previous gut microbiota survey of 15 COVID-19 patients during hospitalization revealed distinct community compositions compared with non-COVID individuals (Zuo et al., 2020), highlighting several gut microbial species that were enriched and depleted in association with COVID-19. Recently, there have been indications that COVID-19 patients develop autoimmune and autoinflammatory symptoms, the most prominent being multisystem inflammatory syndrome and Kawasaki-like disease in children (Cheung et al., 2020b; Galeotti and Bayry, 2020; Verdoni et al., 2020). Since the gut microbiome is intimately involved in the function of human immune systems, it has been hypothesized that gut microbiota is associated with host inflammatory responses in COVID-19. This study reports associations between gut microbiota composition and plasma concentrations of inflammatory markers in 101 COVID-19 patients during hospitalization, as well as a prolonged gut microbiota dysbiosis up to 30 days after negative SARS-CoV-2 quantitative reverse transcription polymerase chain reaction (qRT-PCR) tests.

### Methods

Subject Recruitment and Sample Collection

This study was approved by the Clinical Research Ethics Committee (reference number 2020.076), and all patients provided written informed consent. As described in a previous study (Zuo et al., 2020), COVID-19 patients were recruited from the Prince of Wales and United Christian Hospitals in Hong Kong between February and May 2020. Patients were classified into four severity cohorts based on symptoms as reported by Wu et al., 2020. Briefly, patients were classified as mild if there were no radiographic indications of pneumonia, moderate if pneumonia with fever and respiratory tract symptoms were detected, severe if respiratory rate≥30 breaths per minute, oxygen saturation≤93% when breathing ambient air, or PaO₂/FiO₂≤300 mmHg, critical if respiratory failure requiring mechanical ventilation or organ failure requiring intensive care. Blood and stools from hospitalized patients were collected by hospital staff while discharged patients provided stools during follow-up visits. Samples were stored at −80° C. until processing.

### Stool DNA Extraction and Sequencing

Detailed methods are described in Zuo et al., 2020. Briefly, DNA was extracted from 0.1 g of homogenized fecal samples using the Maxwell RSC PureFood GMO and Authentication Kit and a Maxwell® RSC Instrument nucleic acid extraction platform (Promega, Wisconsin USA) according to manufacturer's instructions. Sequencing libraries were prepared from extracted DNA using the Nextera DNA Flex Library Prep Kit (Illumina, California USA), and sequenced on an Illumina NovaSeq 6000 System at the Centre for Gut Microbiota Research, Chinese University of Hong Kong.

### Sequencing Data Processing, Inferring Gut Microbiota Composition and Statistical Analysis

Raw sequence data were quality filtered using Trimmomatic v0.39 to remove adaptor and low quality sequences. Following this, microbiota composition profiles were inferred from quality-filtered forward reads using MetaPhlAn2 (Truong et al., 2015) v2.7.7 with the v20 database. The site by species counts and relative abundance tables were input into R (R core team, 2018) v3.5.1 for statistical analysis. Principal component analysis (PCA) ordinations were used to visualize the clustering of samples based on their species level compositional profiles. Associations between gut community composition and patients' parameters were assessed using permutational multivariate analysis of variance (PERMANOVA) and Procrustes analyses. Associations of specific microbial species with patient parameters were identified using the linear discriminant analysis effect size (LEfSe) and the multivariate analysis by linear models (MaAsLin) statistical frameworks implemented in the Huttenhower Lab Galaxy instance (website: huttenhower.sph.harvard.edu/galaxy/). PCA, PERMANOVA and Procrustes analysis are implemented in the vegan R package (Oksanen, 2013) v2.4-6.

### Measuring SARS-CoV-2 Load in Stool Samples

SARS-CoV-2 virus loads were measured via reverse transcription quantitative polymerase chain reactions (RT-qPCR) as described in Zuo et al., 2020. RNA was extracted from 0.1 g homogenized stools using the QIAamp Viral RNA Mini Kit (QIAGEN, Hilden Germany) following manufacturer's instructions. SARS-CoV-2 primer and probe sequences were as provided by the US Centers for Disease Control and Prevention (2019-nCoV_N1-F: 5′-GACCCCAAAATCAGC GAAAT-3′ (SEQ ID NO:1), 2019-nCoV_N1-R: 5′-TCTGGTTACTGCCAGTTGAATCTG-3′ (SEQ ID NO:2) and 2019-nCoV_N1-P: 5′-FAM-ACCCCGCATTACGTTTGGTGGACC-BHQ1-3′ (SEQ ID NO:3)). Each one-step RT-qPCR reaction contained 10 ΞL of extracted RNA, 4 μL TaqMan Fast Virus 1-Step Master Mix (Thermo Fisher Scientific, Massachusetts USA) in a final reaction volume of 20 μL. Primer and probe concentrations were 0.5 μM and 0.125 μM, respectively. Cycling conditions were 25° C. for 2 min, 50° C. for 15 min, 95° C. for 2 min, followed by 45 cycles of 95° C. for 15 s and 55° C. for 30 s. Thermocycling was performed on a StepOnePlus Real-Time PCR System (Thermo Fisher Scientific). Cycle threshold (Ct) values were converted into viral RNA copies based on a standard curve prepared from 10-fold serial dilutions of known copies of plasmids containing the full N gene (2019-nCoV_N_Positive Control, Integrated DNA Technologies, USA). Samples were considered negative if Ct values exceeded 39.9 cycles. The detection limit was 347 copies/mL.

### Plasma Cytokine Measurements

Whole blood samples collected in anticoagulant-treated tubes were centrifuged at 2000×g for 10 min and the supernatant was collected. Concentrations of cytokines and chemokines were measured using the MILLIPLEX MAP Human Cytokine/Chemokine Magnetic Bead Panel—Immunology Multiplex Assay (Merck Millipore, Massachusetts USA) on a Bio-Plex 200 System (Bio-Rad Laboratories, California, USA). Concentration of NT-proBNP was measured using Human NT-proBNP ELISA kits (Abcam, Cambridge, UK).

### Data Availability

Raw sequence data generated for this study are available in the Sequence Read Archive under BioProject accession PRJNAXXX.

### Results

### Cohort Description and Study Subjects

Between February and May 2020, 47 females and 53 males with an average age of 36.4±18.7 years (mean±standard deviation; median 32.5, max 75, min 2 years) were recruited for this study. Of the 100 patients, 41 provided multiple stool samples over the duration of their hospital stay and/or follow-up after discharge; 34 and 46 were administered antibiotics and antivirals, respectively, prior to their stool collection. Patients were assigned to disease severity groups according to a retrospective assessment of clinical characteristics in 80 COVID patients. A breakdown of numbers is shown in Table 14.

TABLE 14
Subject characteristics

| | COVID-19 | non-COVID-19 |
|---|---|---|
| | (n = 100) | (n = 78) |
| Male, n (%) | 53 (53.0%) | 33 (42.3%) |
| Years of Age, mean ± SD | 36.4 ± 18.7 | 45.5 ± 13.3 |
| Disease severity category | | |
| Mild | 47 (47.0%) | NA |
| Moderate | 45 (45.0%) | |
| Severe | 5 (5.0%) | |
| Critical | 3 (3.0%) | |
| Symptoms at admission, | | |
| n (%) | | |
| Fever | 38 (38.0%) | NA |
| Diarrhea | 17 (17.0%) | |
| Cough | 40 (40.0%) | |
| Sputum | 18 (18.0%) | |
| Sore throat | 8 (8.0%) | |
| Rhinorrhea | 19 (19.0%) | |
| Shortness of Breath | 9 (9.0%) | |

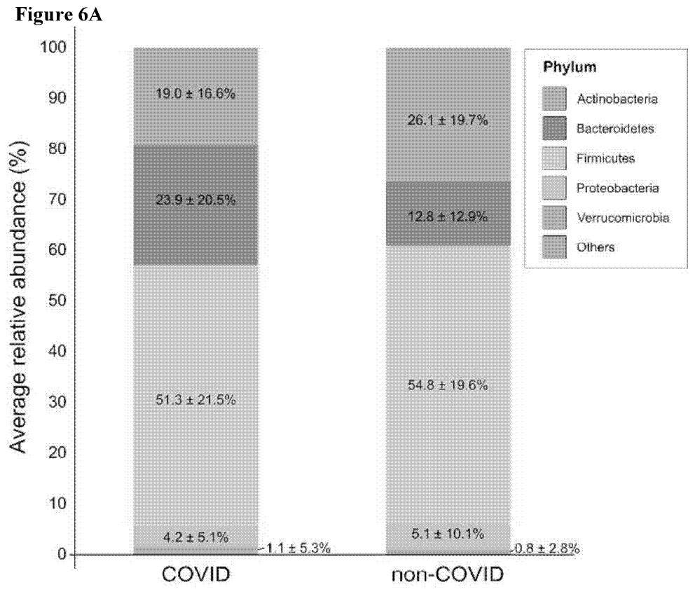

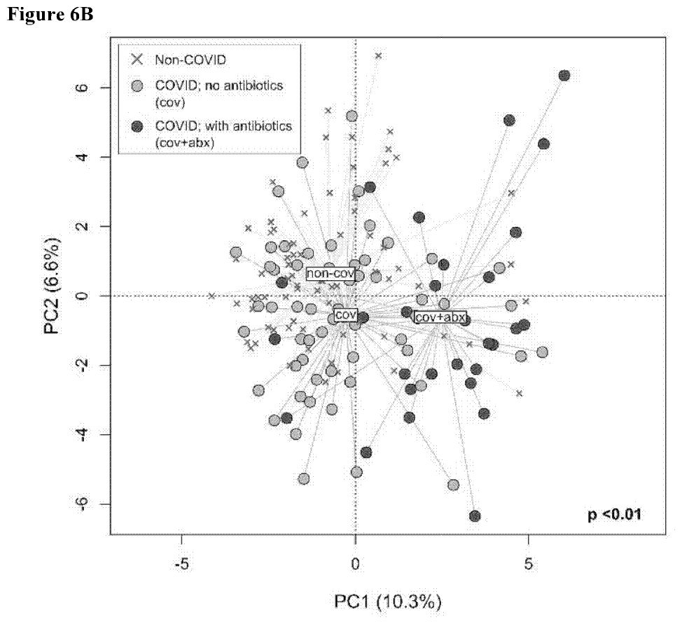

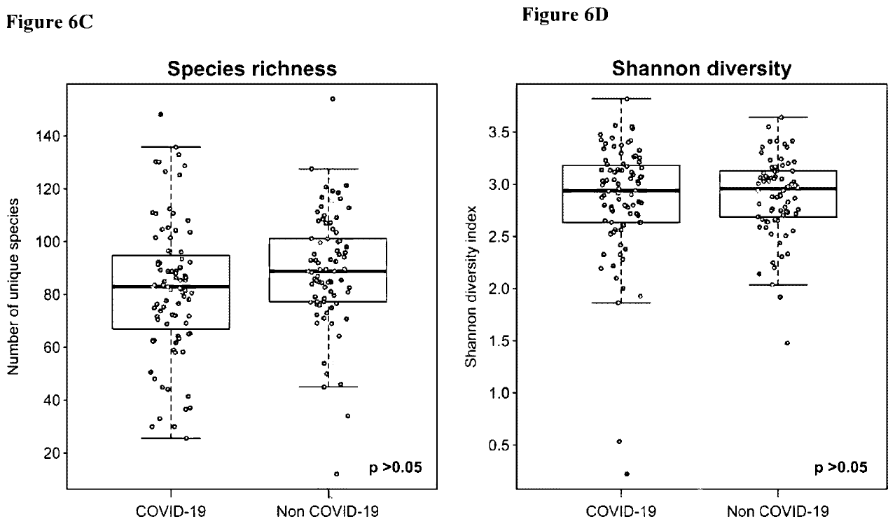

In total, 274 stool samples were sequenced generating an average of 6.8 Gbp (47,386,950 reads) per sample. Firstly, gut microbiota compositions of the first stool samples of each patient collected during hospitalization (n=87) were compared with non COVID-19 gut microbiotas (n=78) obtained from of a Hong Kong cohort to assess whether gut microbiota composition was altered in the COVID-19 patients. At the phylum level, the average gut microbiota of COVID-19 patients was more relatively abundant in Bacteroidetes compared with non COVID-19 subjects (23.9±20.5% vs 12.8±12.9%, p<0.05, Mann Whitney test). Conversely, Actinobacteria were more relatively abundant in the non COVID-19 subjects compared with COVID-19 patients (26.1+19.7 vs 19.0+16.6%%, p <0.05, Mann Whitney test) (FIG. 6A).

Clinical Factors Associated with Species Composition in COVID-19 Patients

When comparing species composition, significant associations were identified with cohort (COVID-19 vs non COVID-19) and antibiotics (FIG. 6B) (p <0.05, PERMANOVA), but not antivirals (Kaletra, Ribavirin or Tamiflu in 39 out of 85 patients), steroids (hydrocortisone in one patient) and proton pump inhibitors (pantoprazole in four patients) (Tables 15 and 16).

Thus, factors listed in Tables 15-16 can be used in different combinations to build a risk assessment model to determine whether a person is at risk of dysbiosis and whether microbiome restoration therapy or supplementation is required. For example, subjects may fill in a questionnaire for collection of information including use of antibiotics, history of COVID-19 and severity of COVID-19, and applied to the data to a computational model to determine the microbial therapy suitable for this subject such as dose and duration.

### Compositional Differences of Gut Bacterial Species in COVID-19 Patients

Without controlling for use of antibiotics in the COVID-19 cohort, compositional differences in the gut were primarily driven by enrichment of species such as Ruminococcus gnavus, Ruminococcus torques and Bacteroides dorei, and depletion of Bifidobacterium adolescentis, Faecalibacterium prausnitzii and Eubacterium rectale in COVID-19 subjects relative to non COVID-19 controls (Linear discriminant analysis Effect Size (LEfSe) (selected taxa with mean relative abundance >1% in either group is shown in (Tables 17-18). When antibiotics was considered, differences between cohorts were primarily linked to enrichment of taxa such as Parabacteroides, Sutterella wadsworthensis and Bacteroides caccae, and depletion of Adlercreutzia equolifaciens, Dorea formicigenerans and Clostridium leptum in patients relative to non COVID-19 controls although most of the implicated taxa comprised less than 0.1% average relative abundance in these samples. While the overall gut microbiota composition was distinct between COVID-19 and non COVID-19 individuals, there were no significant differences in species richness and Shannon diversity when comparing between the COVID-19 and non COVID-19 cohorts (FIG. 6C) (p>0.05, Mann Whitney test).

TABLE 17
Bacterial Species enriched in patients with COVID-19 compared to subjects
without COVID-19

| | | | | mean | |
|---|---|---|---|---|---|
| | | | mean | relative | |
| | | | relative | abundance | |
| | | | abundance | non- | |
| | | Association | COVID-19 | COVID-19 | |
| Species | phylum | cohort | (%) | (%) | NCBI:txid |
| Ruminococcus gnavus | Birmicutes | COVID | 4.64 | 1.82 | 33038 |
| Ruminococcus torques | Birmicutes | COVID | 4.44 | 2.27 | 33039 |
| Bacteroides dorei | Bacteroidetes | COVID | 3.03 | 0.74 | 357276 |
| Bacteroides vulgatus | Bacteroidetes | COVID | 2.84 | 1.14 | 821 |
| Bacteroides ovatus | Bacteroidetes | COVID | 1.92 | 0.62 | 28116 |
| Bacteroides caccae | Bacteroidetes | COVID | 1.46 | 0.41 | 47678 |
| Akkermansia muciniphila | Verrucomicrobia | COVID | 1.06 | 0.77 | 239935 |

TABLE 18
Bacterial Species enriched in subjects without COVID-19 compared to
patients with COVID-19

| | | | | mean | |
|---|---|---|---|---|---|
| | | | mean | relative | |
| | | | relative | abundance | |
| | | | abundance | non- | |
| | | Association | COVID-19 | COVID-19 | |
| Species | phylum | cohort | (%) | (%) | NCBI:txid |
| Bifidobacterium | Actinobacteria | non | 3.94 | 7.78 | 1680 |
| adolescentis | | COVID | | | |
| Eubacterium rectale | Firmicutes | non | 3.14 | 6.78 | 39491 |
| | | COVID | | | |
| Faecalibacterium | Firmicutes | non | 3.69 | 5.89 | 853 |
| prausnitzii | | COVID | | | |
| Ruminococcus bromii | Firmicutes | non | 2.19 | 5.73 | 40518 |
| | | COVID | | | |
| Subdoligranulum | Firmicutes | non | 2.39 | 4.90 | 2685293 |
| unclassified | | COVID | | | |
| Collinsella aerofaciens | Actinobacteria | non | 2.58 | 4.49 | 74426 |
| | | COVID | | | |
| Bifidobacterium | Actinobacteria | non | 1.94 | 3.83 | 28026 |
| pseudocatenulatum | | COVID | | | |
| Ruminococcus obeum | Firmicutes | non | 1.69 | 2.40 | 40520 |
| | | COVID | | | |
| Dorea formicigenerans | Firmicutes | non | 1.35 | 1.53 | 39486 |
| | | COVID | | | |
| Dorea longicatena | Firmicutes | non | 1.09 | 1.50 | 88431 |
| | | COVID | | | |
| Coprococcus comes | Firmicutes | non | 0.99 | 1.37 | 410072 |
| | | COVID | | | |

Thus, bacterial species listed in Tables 17-18 can be used in different combinations to build a risk assessment model to determine whether a person is at risk of dysbiosis and whether microbiome restoration therapy or supplementation is required.

For prevention, treatment, or facilitation of recovery of COVID-19, bacterial species listed in Table 17 may be suppressed in a COVID-19 patient or one who has been exposed to the coronavirus and at risk of becoming infected to a level lower than or equal to the mean relative abundance of non COVID-19 subjects. Such suppression can be induced by administration of an effective amount of one or more bacteria listed in Table 17 or bacteria with a positive correlation with the bacteria listed in Table 17, such as by FMT.

Also for prevention, treatment or facilitation of recovery of COVID-19, bacterial species listed in Table 18 may be increased in a COVID-19 patient or one who has been exposed to the coronavirus and is at risk of suffering from COVID-19 to a level higher than or equal to the mean relative abundance of non-COVID-19 subjects. Such increase or enhancement can be achieved by administration of bacteria having negative correlation with one or more of the bacterial species listed in Table 18 including by FMT.

Plasma CXCL10, IL-10, and TNF-α Concentrations are Associated with Gut Microbiota Composition

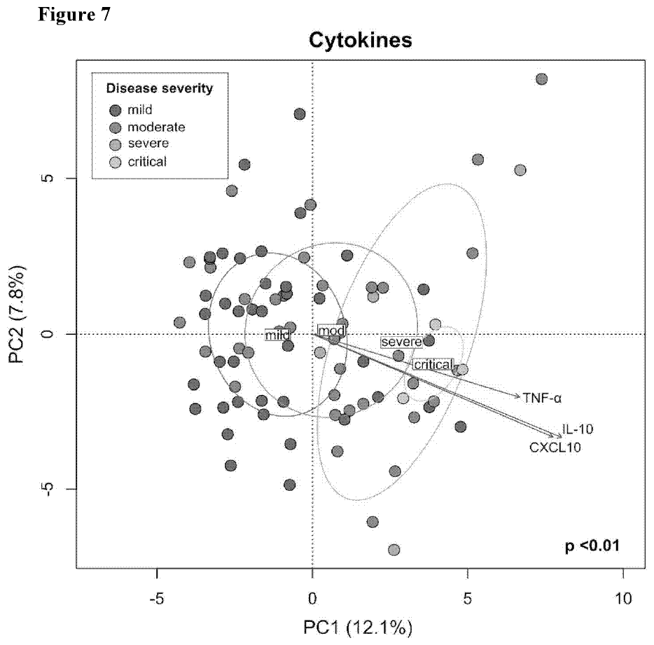

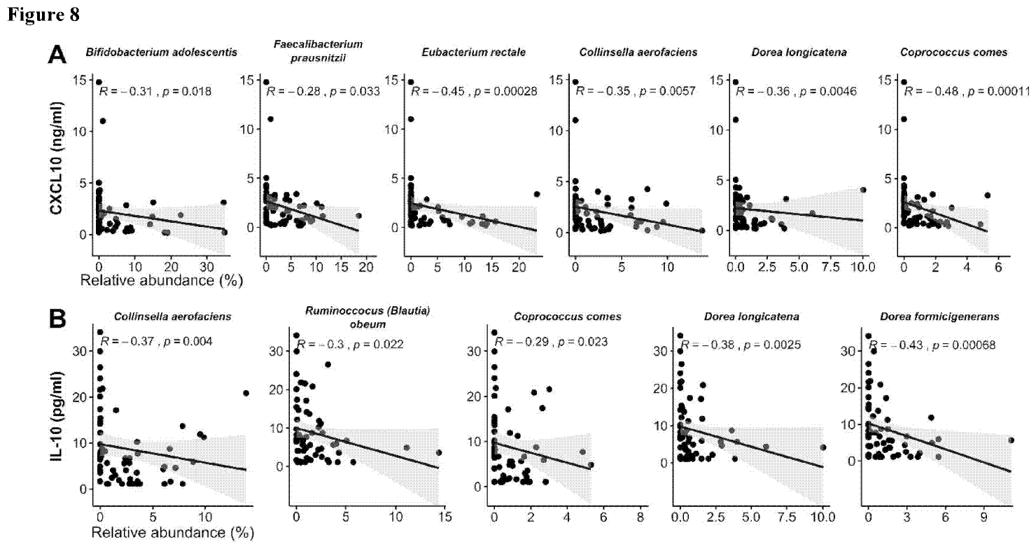

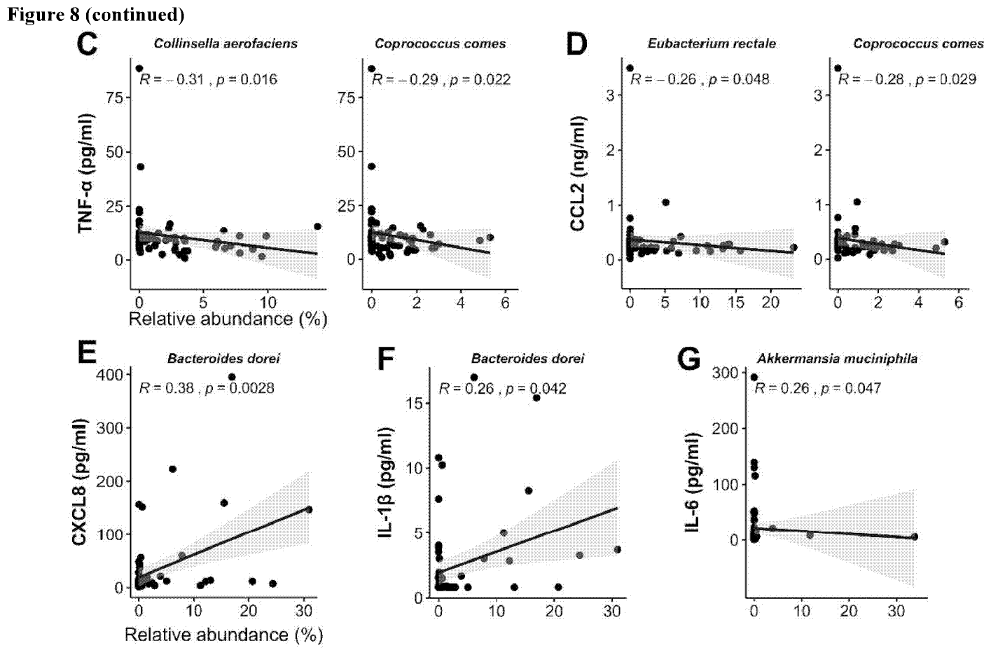

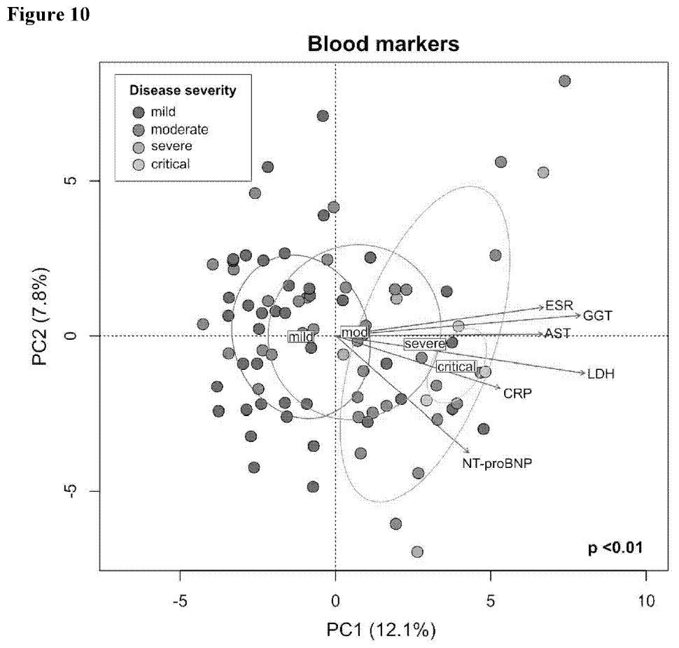

In COVID-19, the immune system produces inflammatory cytokines in response to virus infection. In some cases, the response can be overaggressive (i.e., cytokine storm) and results in widespread tissue damage, septic shock and multiple organ failure (Tay et al., 2020). Based on the observation that the gut microbiota is altered in COVID-19 patients, it has been hypothesized that these compositional changes play a role in exacerbating disease by contributing to dysregulation of the immune response. Within the COVID-19 cohort, principal component analysis (PCA) visualization of gut microbiota composition data revealed a continuum along the mild, moderate, severe, and critical disease severity groups (p<0.05, PERMANOVA) (FIG. 7), indicating a likely stratification of gut microbiota composition associated with disease severity. As plasma concentrations of cytokines and inflammatory markers were fitted onto the PCA, it was observed that CXCL10, IL-10, and TNF-α were significantly associated with gut microbiota composition (p<0.05, Procrustes analysis), notably their values increased concomitant with disease severity (FIG. 7, FIG. 10). Since CXCL10, IL-10, and TNF-α are typically elevated in COVID-19 (Vabret et al., 2020), these results indicate that gut microbiota composition is associated with the magnitude of immune response to COVID-19 and can play a role in regulating disease severity. It was then specifically assessed as to which microbial species were enriched/depleted in the COVID-19 cohort and whether they were correlated with plasma cytokine concentrations. From the list of most relatively abundant species in Tables 12 and 13, six COVID-19 depleted species were negatively correlated with CXCL10, five with IL-10, and two each with TNF-α and CCL2 (FIG. 8A-8D, Table 19). These included species such as Bifidobacterium adolescentis, Bilidobacterium bifidum, Eubacterium rectale and Faecalibacterium prausnitzii known to play immunomodulatory roles in the human GI system. Conversely, only two COVID-19 enriched species Bacteroides dorei and Akkermansia muciniphila were positively correlated with IL-113, IL-6 and CXCL8 (FIG. 8E-8G).

For prevention of development of severe disease in COVED-19, bacterial species listed in Table 19, especially Bifidobacterium adolescentis, Bifidobacterium bifidum, Eubacterium rectale and Faecalibacterium prausnitzii, should be increased in a subject already diagnosed with COVID-19 but does not yet have severe symptoms to a level higher than or equal to the mean relative abundance of non-COVID-19 subjects. Such increase or enhancement can be achieved by administration of one or more bacteria species listed in Table 19 or bacteria positively correlated with one or more bacteria species listed in Table 19, for example, by FMT.

Prolonged Gut Microbiota Dysbiosis after Recovery from COVID-19

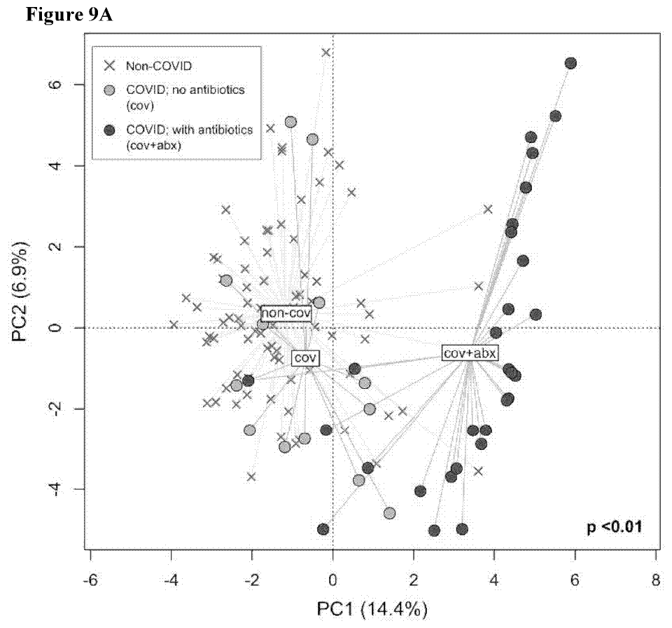

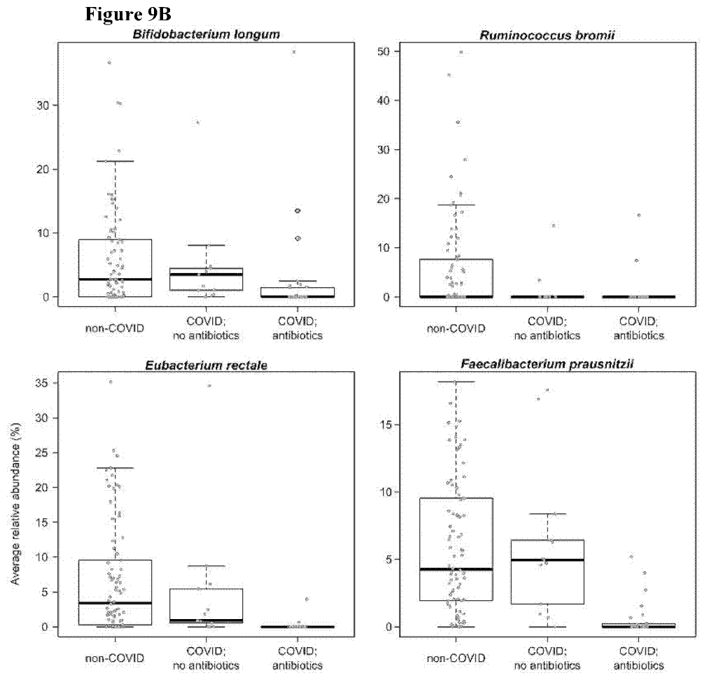

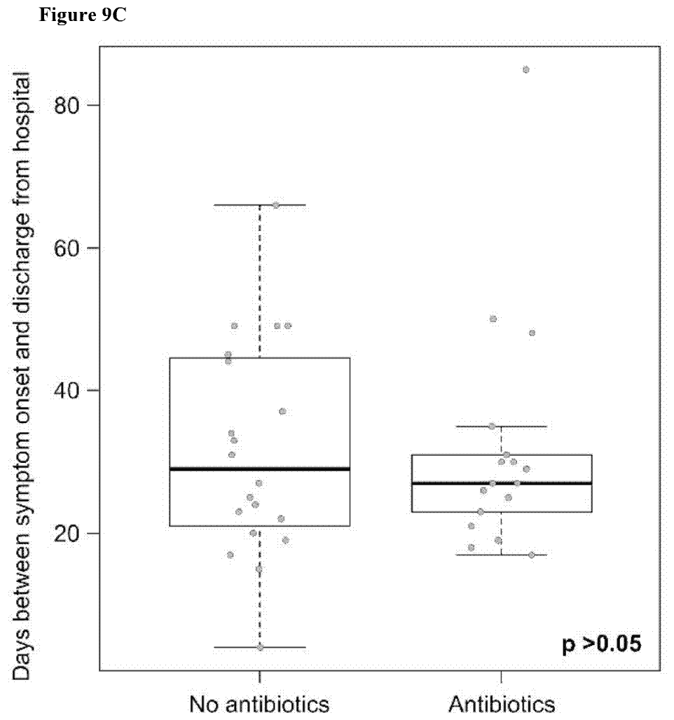

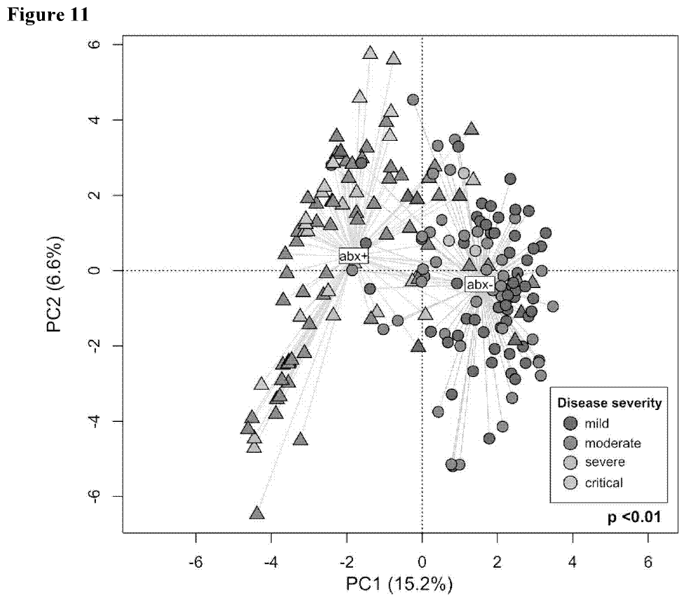

To assess gut microbiota composition following recovery from COVID-19, 42 stool samples were collected from 27 patients after their nasopharyngeal aspirates or swabs tested negative for SARS-COV-2 via qRT-PCR. Compared with non COVID-19 controls, gut microbiota composition of recovered patients, some profiled up to 30 days (median six days, interquartile range 14 days) after negative qRT-PCRs remained significantly distinct irrespective of whether patients received antibiotics or not (p<0.05, PERMANOVA). Moreover, the gut microbiota of patients who had received antibiotics were more dissimilar than patients who did not receive antibiotics when compared with non COVID-19 controls (FIG. 9A), indicating that the influence of antibiotics on gut microbiota composition persists after recovery from COVID-19 (FIG. 11). Gut microbiotas of recovered patients were enriched in species such as Bifidobacterium dentium and Lactobacillus ruminis, and depleted in Eubacterium rectale, Ruminococcus bromii, Faecalibacterium prausnitzii and Bifidobacterium longum (FIG. 9B) (Tables 20 and 21). The depletion was even more striking in recovered patients who had received antibiotics with several of these taxa recording more than an order of magnitude decrease in relative abundance compared with non-antibiotics patients. As to whether antibiotics was associated with improved disease outcomes, its use in the moderate severity cohort (21 out of 45 received antibiotics) was examined, and no statistical difference was found in the number of days from onset of COVID-19 symptoms until discharge from hospital with or without antibiotics (FIG. 9C) (p>0.05, Mann-Whitney test). As there were no records of bacteraemia in the 45 patients, this finding indicate that antibiotics are unlikely to result in improved patient outcomes in COVID-19 assuming no bacterial co-infections.

For prevention, treatment or facilitation of recovery of COVID-19, bacterial species listed in Table 20 such as Bifidobacterium dentium and Lactobacillus ruminis should be suppressed in COVID-19 patients to a level lower than or equal to the mean relative abundance of non COVID-19 subjects. Such suppression can be achieved by administration of bacteria that have negative correlation with the bacteria listed in Table 20, such as by FMT.

For prevention, treatment or facilitation of recovery of COVID-19, bacterial species listed in Table 21 especially Eubacterium rectale, Ruminococcus bromii, Faecalibacterium prausnitzii and Bifidobacterium longum should be increased in COVID-19 patients to a level higher than or equal to the mean relative abundance of non-COVID-19 subjects. Such increase or enhancement can be achieved by administration of an effective amount of one or more of the bacteria species listed in Table 21 or bacteria having positive correlation with the bacteria listed in Table 21, for example, by FMT.

### REFERENCES

Cheung, E. W., P. Zachariah, M. Gorelik, A. Boneparth, S. G Kerrie, J. S. Orange and J. D. Milner (2020). “Multisystem Inflammatory Syndrome Related to COVID-19 in Previously Healthy Children and Adolescents in New York City.” JAMA.
Cheung, K. S., I. F. N. Hung, P. P. Y Chan, K. C. Lung, E. Tso, R. Liu, Y. Y Ng, M. Y. Chu, T. W. H. Chung, A. R. Tam, C. C. Y. Yip, K. H. Leung, A. Y. Fung, R. R. Zhang, Y. Lin, H. M. Cheng, A. J. X. Zhang, K. K. W. To, K. H. Chan, K. Y. Yuen and W. K. Leung (2020). “Gastrointestinal Manifestations of SARS-CoV-2 Infection and Virus Load in Fecal Samples From a Hong Kong Cohort: Systematic Review and Meta-analysis.” Gastroenterology 159(1): 81-95.
Galeotti, C. and J. Bayry (2020). “Autoimmune and inflammatory diseases following COVID-19.” Nat Rev Rheumatol 16(8): 413-414.
Lamers, M. M., J. Beumer, J. van der Vaart, K. Knoops, J. Puschhof, T. I. Breugem, R. B. G Ravelli, J. Paul van Schayck, A. Z. Mykytyn, H. Q. Duimel, E. van Donselaar, S. Riesebosch, H. J. H. Kuijpers, D. Schipper, W. J. van de Wetering, M. de Graaf, M. Koopmans, E. Cuppen, P. J. Peters, B. L. Haagmans and H. Clevers (2020). “SARS-CoV-2 productively infects human gut enterocytes.” Science 369(6499): 50-54.
Oksanen, J., F. G Blanchet, R. Kindt, P. Legendre, P. Minchin, R. B. O'Hara, G Simpson, P. Solymos, M. H. N. Stevens and H. Wagner (2012). “Package ‘vegan’—Community Ecology Package, version 2.0-4.”
R Core Team (2018). “R: A Language and Environment for Statistical Computing.”
Tay, M. Z., C. M. Poh, L. Renia, P. A. MacAry and L. F. P. Ng (2020). “The trinity of COVID-19: immunity, inflammation and intervention.” Nat Rev Immunol 20(6): 363-374.
Truong, D. T., E. A. Franzosa, T. L. Tickle, M. Scholz, G Weingart, E. Pasolli, A. Tett, C. Huttenhower and N. Segata (2015). “MetaPhlAn2 for enhanced metagenomic taxonomic profiling.” Nat Methods 12(10): 902-903.
Vabret, N., G J. Britton, C. Gruber, S. Hegde, J. Kim, M. Kuksin, R. Levantovsky, L. Malle, A. Moreira, M. D. Park, L. Pia, E. Risson, M. Saffern, B. Salome, M. Esai Selvan, M. P. Spindler, J. Tan, V. van der Heide, J. K. Gregory, K. Alexandropoulos, N. Bhardwaj, B. D. Brown, B. Greenbaum, Z. H. Gumus, D. Homann, A. Horowitz, A. O. Kamphorst, M. A. Curotto de Lafaille, S. Mehandru, M. Merad, R. M. Samstein and P. Sinai Immunology Review (2020). “Immunology of COVID-19: Current State of the Science.” Immunity 52(6): 910-941.
Verdoni, L., A. Mazza, A. Gervasoni, L. Martelli, M. Ruggeri, M. Ciuffreda, E. Bonanomi and L. D'Antiga (2020). “An outbreak of severe Kawasaki-like disease at the Italian epicentre of the SARS-CoV-2 epidemic: an observational cohort study.” Lancet 395(10239): 1771-1778.
Vetter, P., D. L. Vu, A. G L'Huillier, M. Schibler, L. Kaiser and F. Jacquerioz (2020). “Clinical features of covid-19.” BMJ 369: m1470.
Wolfel, R., V. M. Corman, W. Guggemos, M. Seilmaier, S. Zange, M. A. Muller, D. Niemeyer, T. C. Jones, P. Vollmar, C. Rothe, M. Hoelscher, T. Bleicker, S. Brunink, J. Schneider, R. Ehmann, K. Zwirglmaier, C. Drosten and C. Wendtner (2020). “Virological assessment of hospitalized patients with COVID-2019.” Nature 581(7809): 465-469.
Wu, J., J. Liu, X. Zhao, C. Liu, W. Wang, D. Wang, W. Xu, C. Zhang, J. Yu, B. Jiang, H. Cao and L. Li (2020). “Clinical Characteristics of Imported Cases of Coronavirus Disease 2019 (COVID-19) in Jiangsu Province: A Multicenter Descriptive Study.” Clin Infect Dis 71(15): 706-712.
Xu, Y., X. Li, B. Zhu, H. Liang, C. Fang, Y Gong, Q. Guo, X. Sun, D. Zhao, J. Shen, H. Zhang, H. Liu, H. Xia, J. Tang, K. Zhang and S. Gong (2020). “Characteristics of pediatric SARS-CoV-2 infection and potential evidence for persistent fecal viral shedding.” Nat Med 26(4): 502-505.
Zuo, T., F. Zhang, G C. Y Lui, Y K. Yeoh, A. Y. L. Li, H. Zhan, Y Wan, A. Chung, C. P. Cheung, N. Chen, C. K. C. Lai, Z. Chen, E. Y K. Tso, K. S. C. Fung, V. Chan, L. Ling, G Joynt, D. S. C. Hui, F. K. L. Chan, P. K. S. Chan and S. C. Ng (2020). “Alterations in Gut Microbiota of Patients With COVID-19 During Time of Hospitalization.” Gastroenterology.

The contents of all patents, patent applications, and other publications, including GenBank Accession Numbers or the equivalent, cited in this application are incorporated by reference in the entirety for all purposes.

What is claimed is:

1. A method for treating COVID-19 symptoms or facilitating recovery from COVID-19 in a human subject infected by severe acute respiratory syndrome coronavirus 2 (SARS-CoV-2), comprising introducing into the subject's gastrointestinal tract an effective amount of (i) bacterial species Bacteroides dorei, or one or more of the bacterial species set forth in Tables 4, 5, 6, 9, 13, and 18 or belonging to any one of the bacterial taxa set forth in Tables 19 and 21; or (ii) one or more of the viral species set forth in Table 11.

2. The method of claim 1, wherein the introducing step comprises oral administration to the subject a composition comprising an effective amount of the bacterial species or viral species.

3. The method of claim 1, wherein the introducing step comprises delivery to the small intestine, ileum, or large intestine of the subject a composition comprising an effective amount of the bacterial species or viral species.

4. The method of claim 1, wherein the introducing step comprises fecal microbiota transplantation (FMT).

5. The method of claim 4, wherein the FMT comprises administration to the subject a composition comprising processed donor fecal material.

6. The method of claim 1, wherein the introducing step further comprises simultaneously introducing to the subject a prebiotic or a therapeutic agent effective for treating COVID-19.

7. The method of claim 6, wherein the prebiotic or therapeutic agent is introduced in the same composition comprising the effective amount of the bacterial species or viral species.

8. The method of claim 2, wherein the composition is administered before and/or with food intake.

9. The method of claim 1, wherein the level or relative abundance of the bacterial species or viral species is determined in a first stool sample obtained from the subject prior to the introducing step and in a second stool sample obtained from the subject after the introducing step.

10. The method of claim 9, wherein the level of the bacterial species or viral species is determined by quantitative polymerase chain reaction (PCR).

11. The method of claim 1, wherein the one or more bacterial species comprise Bifidobacterium adolescentis, Faecalibacterium prausnitzii, Eubacterium rectale, Bifidobacterium bifidum, or Bifidobacterium longum.

12. A method for treating COVID-19 symptoms or facilitating recovery from COVID-19 in a human subject infected by SARS-CoV-2, comprising reducing the level or relative abundance of (i) one or more of the bacterial species set forth in Tables 3, 7, 8, 12, and 17 or belonging to any one of the bacterial taxa set forth in Table 20; or (ii) one or more of the viral species set forth in Table 10 in the subject's gastrointestinal tract.

13. The method of claim 12, wherein the reducing step comprising FMT.

14. The method of claim 13, wherein the reducing step comprises treating the subject with an anti-bacterial or anti-viral agent.

15. The method of claim 14, wherein a composition comprising processed donor fecal material is introduced to the gastrointestinal tract of the subject after the subject is treated with the anti-bacterial or anti-viral agent.

16. The method of claim 12, further comprising simultaneously administering to the subject a prebiotic or a therapeutic agent effective for treating COVID-19.

17. The method of claim 16, wherein the prebiotic or therapeutic agent is orally administered.

18. The method of claim 12, wherein the level or relative abundance of the bacterial species or the viral species is determined in a first stool sample obtained from the subject prior to the reducing step and in a second stool sample obtained from the subject after the reducing step.

19. The method of claim 18, wherein the level of the bacterial species or viral species is determined by quantitative polymerase chain reaction (PCR).

20. The method of claim 12, wherein the one or more bacterial species comprise Bifidobacterium dentium or Lactobacillus ruminis.

21. A kit comprising: a first container containing a first composition comprising (i) an effective amount of bacterial species Bacteroides dorei or one or more of the bacterial species set forth in Tables 4, 5, 6, 9, 13, and 18 or belonging to any one of the bacterial taxa set forth in Tables 19 and 21, (ii) an effective amount of one or more of the viral species set forth in Table 11, (iii) an effective amount of an anti-bacterial agent that suppresses growth of one or more of the bacterial species set forth in Tables 3, 7, 8, 12, and 17 or belonging to any one of the bacterial taxa set forth in Table 20, or (iv) an effective amount of an anti-viral agent that suppresses growth of one or more of the viral species set forth in Table 10, and a second container containing a second composition comprising a prebiotic or a therapeutic agent effective for treating COVID-19.

22. The kit of claim 21, wherein the first composition comprises processed donor fecal material for FMT.

23. The kit of claim 21 or 22, wherein the first composition is formulated for oral administration.

24. The kit of claim 21, wherein the second composition is formulated for oral administration.

25. The kit of claim 21, wherein both the first and second compositions are formulated for oral ingestion.

26. The kit of claim 23, wherein the one or more bacterial species comprise Bifidobacterium adolescentis, Faecalibacterium prausnitzii, Eubacterium rectale, Bifidobacterium bifidum, or Bifidobacterium longum

27. A method for predicting severity of COVID-19 among human subjects who have been infected by SARS-CoV-2, comprising:
(1) determining, in a stool sample from a first human subject infected by SARS-CoV-2, the level or relative abundance of any one of the bacterial species set forth in Tables 6, 9, 13, and 18 or belonging to any one of the bacterial taxa set forth in Tables 19 and 21, or the level or relative abundance of any one of the viral species set forth in Table 11;
(2) detecting the level of relative abundance from step (1) being higher than the level or relative abundance of the same bacterial or viral species in a stool sample from a second human subject infected by SAES-CoV-2; and
(3) determining the second subject as likely to experience more severe COVID-19 than the first subject.

28. The method of claim 27, wherein the level or relative abundance of multiple bacterial species set forth in Tables 6, 9, 13, and 18 or belonging to the bacterial taxa set forth in Tables 19 and 21 or multiple viral species set forth in Table 11 is determined, and the level or of more than half of the multiple bacterial species or viral species in the first subject's sample is higher than the corresponding level or relative abundance in the second subject's sample, and the second subject is determined to likely experience more severe COVID-19 than the first subject.

29. The method of claim 27, wherein the bacterial species is Bifidobacterium adolescentis, Faecalibacterium prausnitzii, Eubacterium rectale, Bifidobacterium bifidum, or Bifidobacterium longum.

30. A method for predicting severity of COVID-19 among human subjects who have been infected by SARS-CoV-2, comprising:
(1) determining, in a stool sample from a first human subject infected by SARS-CoV-2, the level or relative abundance of one or more of the bacterial species set forth in Tables 7, 8, 12, and 17 or belonging to one or more of the bacterial taxa set forth in Table 20, or the level or relative abundance of one or more of the viral species set forth in Table 10;
(2) detecting the level of relative abundance from step (1) being higher than the level or relative abundance of the same bacterial or viral species in a stool sample from a second human subject infected by SARS-CoV-2; and
(3) determining the first subject as likely to experience more severe COVID-19 than the second subject.

31. The method of claim 30, wherein the level or relative abundance of multiple bacterial species set forth in Tables 7, 8, 12, and 17 or belonging to the bacterial taxa set forth in Table 20 or the level or relative abundance of multiple viral species set forth in Table 10 is determined, and the level or of more than half of the multiple bacterial or viral species in the first subject's sample is higher than the corresponding level or relative abundance in the second subject's sample, and the first subject is determined to likely experience more severe COVID-19 than the second subject.

32. The method of claim 30, wherein the bacterial species is Bifidobacterium dentium or Lactobacillus ruminis

33. The method of any one of claims 27-32, wherein the level or relative abundance of the bacterial or viral species is determined by quantitative PCR.

34. The method of claims 27-29, further comprising the step of administering to the second subject an effective amount of a therapeutic agent effective for treating COVID-19.

35. The method of claim 30 or 31, further comprising the step of administering the first subject an effective amount of a therapeutic agent effective for treating COVID-19.

36. A kit for assessing COVID-19 severity in a patient, comprising a set of oligonucleotide primers for amplification of a polynucleotide sequence from (1) any one of the bacterial species set forth in Tables 6, 7, 8, 9, 12, 13, 17, and 18 or belonging to any one of the bacterial taxa set forth in Tables 19-21, or (2) any one of the viral species set forth in Tables 10 and 11.

37. The kit of claim 36, wherein the amplification is PCR.

38. The kit of claim 37, further comprising reagents for quantitative PCR.

39. The kit of claim 36, wherein the bacterial species is Bifidobacterium adolescentis, Faecalibacterium prausnitzii, Eubacterium rectale, Bifidobacterium bifidum, or Bifidobacterium longum.

40. The kit of claim 36, the bacterial species is Bifidobacterium dentium or Lactobacillus ruminis.
